(19) United States
(12) Patent Application Publication
(10) Pub. No.: US 2020/0129665 A1
(43) Pub. Date: Apr. 30, 2020

(54) NOVEL NANOFIBER-BASED GRAFTS AND METHODS OF MAKING AND USING THE SAME

(71) Applicant: WAKE FOREST UNIVERSITY HEALTH SCIENCES, Winston-Salem, NC (US)

(72) Inventors: William D. Wagner, Clemmons, NC (US); Harleigh J. Warner, Myrtle Beach, SC (US)

(21) Appl. No.: 16/493,458

(22) PCT Filed: Apr. 4, 2018
(86) PCT No.: PCT/US18/26050

§ 371 Date: Sep. 12, 2019

Related U.S. Application Data
(60) Provisional application No. 62/481,403, filed on Apr. 4, 2017.

(51) Int. Cl. A61L 27/26; A61L 27/38; A61L 27/50

(52) U.S. Cl. CPC A61L 27/26; A61L 27/3804; A61L 2430/20; A61L 2400/12; A61L 27/507

(57) ABSTRACT

Nanofiber-based biomaterials containing fibroin for wound repair and tissue replacement are provided with methods of making and using the same.

This application claims the benefit of U.S. Provisional Patent Application Ser. No. 62/481,403, filed Apr. 4, 2017, the disclosure of which is incorporated herein in their entirety by reference.

### FIELD OF THE INVENTION

The present invention relates generally to electrospun nanofiber-based biomaterials useful for wound repair and tissue replacement and, more particularly, but not exclusively to electrospun composite materials comprising fibroin for tissue engineering applications.

### BACKGROUND OF THE INVENTION

Severe valvular heart disease (VHD) affects 1 out of 40 adults in the United States, and is responsible for approximately 28,000 deaths per year. Aortic valves consist of 3 film-like cusps with an average thickness of 300-700 μm. The main composition of the valvular extracellular material (ECM) is type I collagen and elastin in a 4:1 dry weight ratio which orchestrate the passive opening and closing of the aortic leaflets to direct blood flow. Dysfunctional heart valves are life-threatening as the diseased valvular tissues are unable to perform the normal physiological requirements. Among all VHD, aortic valve disease has a mortality rate of about 65%. The treatment usually necessitates surgical replacement by mechanical or tissue bioprosthetic valves. Commonly used mechanical heart valves have adequate durability but are often thrombogenic and require life-long anti-coagulant therapy. Bioprosthetic collagen-based tissue valves from porcine valves or bovine pericardium mimic the anatomy of native valves, however, early valve degeneration, and 50% postoperative failure occur within 12-15 years. Therefore, a nonthrombogenic and durable alternative is desperately needed in the field.

Successful heart valve grafts should be both durable and functional. Basic anatomical and physiological requirements need to be considered to fabricate structurally similar and mechanically robust synthetic heart valve grafts. Approaches to overcome the pathological failure modes should be taken into consideration in order to select graft composites that are biocompatible, slowly degradable and durable, capable of promoting adequate cell growth and tissue remodelling, while being non-thrombogenic.

### SUMMARY OF THE INVENTION

The present invention provides a nonthrombogenic and durable alternative biomaterial for tissue replacement. In the present invention, collagen protein is blended with mechanically robust fibroin and a hemocompatible synthetic elastomeric polymer or elastomer to produce a multi-functional electrospun nanofibrous material suitable for tissue replacement and specifically, although not exclusively, heart valve or vascular replacement.

In a first aspect, the present invention encompasses a nonthrombogenic composition comprising collagen, fibroin, a hemocompatible synthetic elastomer, and extracellular matrix (ECM) and/or a heparan sulphate glycoprotein. In a particular embodiment, the collagen is type I collagen. In a particular embodiment, the fibroin is silk fibroin. In a particular embodiment, the hemocompatible synthetic elastomer is a polyglycerol derivative ester comprising a polycarboxylic acid. In a particular embodiment, the polyglycerol derivative is poly (glycerol sebacate) (PGS).

In a particular embodiment, the composition comprising collagen, fibroin, and a hemocompatible synthetic elastomer is conditioned with cellular extracellular matrix. In a particular embodiment, the electrospun fiber is covalently linked to a heparan sulphate glycoprotein (e.g., heparan sulphate syndecan; e.g., syndecan 4).

In an additional aspect, the present invention encompasses an article of manufacture comprising collagen, fibroin, a hemocompatible synthetic elastomer in fiber form, and ECM and/or a heparan sulphate glycoprotein.

In another aspect, the present invention encompasses a porous electrospun graft material which is readily configured to function as a tissue replacement. The graft comprises a collagen, a fibroin, a hemocompatible synthetic elastomer, and ECM and/or a heparan sulphate glycoprotein. The porosity of the graft material may have variable degrees of porosity (e.g., to allow different degrees of infiltration). In one embodiment, the tissue to be replaced is heart tissue or vascular tissue. In another embodiment, the graft is molded in the shape of a heart valve.

In yet another aspect, the present invention encompasses a graft for heart valve replacement, comprising a porous electrospun mat, the electrospun mat comprising polyglycerol polymer, fibroin, a polyglycerol derivative ester comprising a poly carboxylic acid, and ECM and/or a heparan sulphate glycoprotein.

In an additional aspect, the present invention encompasses a method of preparing a graft (e.g., for heart valve or arterial replacement), where the method comprises the steps of: (a) electrospinning an electrospinning solution into an interconnected nanofiber matrix; (b) conditioning the interconnected nanofiber matrix with ECM; and (c) collecting the interconnected nanofiber matrix to produce the graft (e.g., for heart valve or arterial replacement). In a particular embodiment, the method further comprises preparing an electrospinning solution of collagen, fibroin and a hemocompatible synthetic elastomer. In a particular embodiment, step (b) comprises culturing the interconnected nanofiber matrix with cells which produce extracellular matrix (e.g., fibroblast or human aortic smooth muscle cells (HASMCs)). In a particular embodiment, the extracellular matrix producing cell is a fibroblast. In a particular embodiment, the interconnected nanofiber matrix is cultured with the cells which produce extracellular matrix for about 1 to about 30 days, particularly about 3 to about 20 days, about 5 to about 15 days, or about 10 days. In a particular embodiment, the method further comprises lysing the cultured cells by delivering a lysing agent to the cells cultured with the interconnected nanofiber matrix (e.g., before or after (c)). Examples of lysing agents include, without limitation, detergents and chaotropic agents. Chaotropic agents include, but are not limited to, guanidinium hydrochloride, guanidinium thiocyanate, guanidinium isothiocyanate and sodium iodide. Chaotropic salts can also be used which comprise a chaotropic anion selected form the group consisting of trichloroacetate, perchlorate and trifluoroacetate. Detergents can be ionic and/or non-ionic detergents. In a particular embodiment, a non-ionic detergent such as but not limited to Triton X100, Tween, Brij35 or NP-40 is used.

The present invention provides electrospun grafts having superior mechanical properties, low degradation, and reduced thrombogenic potential compared to primarily collagen-based materials.

### BRIEF DESCRIPTION OF THE DRAWINGS

The foregoing summary and the following detailed description of the exemplary embodiments of the present invention may be further understood when read in conjunction with the appended drawings, in which:

FIG. 1 depicts a Fourier-transform infrared (FTIR) spectra of collagen, PGS, fibroin and composites thereof.

FIGS. 2A-2E depict scanning electron microscopic images illustrating the morphologies of certain electrospun fibers. The diameters of the random arrays of electrospun crosslinked fibers after crosslinking, and treatment with glycine and water are shown. (FIG. 2A) Collagen:PGS (9:1); (FIG. 2B) collagen:fibroin:PGS (8:1:1); (FIG. 2C) collagen:fibroin:PGS (4.5:4.5:1); (FIG. 2D) collagen:fibroin:PGS (1:8:1); (FIG. 2E) fibroin: PGS (9:1). With higher silk fibroin protein content, smaller fiber diameters were observed (Magnification 2300×).

FIG. 3 depicts differential scanning calorimetry (DSC) scans of electrospun mats prepared from collagen, PGS, fibroin and composites thereof.

FIG. 4 illustrates the forces required to tear sutures made from electrospun mats and porcine heart valves. Duplicate samples were tested for each material. Collagen:Fibroin:PGS (4.5:4.5:1) showed the highest suture pull-out strength with a maximum average load of 0.32 N as compared to the maximum average load of 0.64 N for fresh porcine heart valve.

FIG. 5 illustrates degradation of a PGS-fibroin-collagen (PFC) mat during a 30 week period. Data points are presented as mean±SEM (n=4).

FIG. 6 illustrates cell numbers for HUVEC cultured 3 or 7 days on culture dishes and PFC mats. Bars represent the mean±SEM for each observation from representative photomicrographs (n=4). An increase number of HUVECs was observed from day 3 to day 7. For cells on both substrates, cell numbers increased significantly from day 3 to day 7 (p<0.05).

FIGS. 7A-7E display a number of confocal microscopic images illustrating HUVEC morphology and proliferation on: FIG. 7A: PFC mat at day 3 (images 1 and 2 represent the combined confocal image, 3 represents only the cell nuclei), FIG. 7B: PFC mat at day 7 (images 1 and 2 represent the combined confocal image, 3 represents only the cell nuclei), FIG. 7C: collagen mat at day 3 (images 1 and 2 represent the combined confocal image, 3 represent only the cell nuclei), FIG. 7D: collagen mat at day 7 (images 1 and 2 represents the combined confocal image, 3 represent only the cell nuclei) (cells were seeded at 50,000 per dish on a 48-well plate, representative photomicrographs depict increased cell numbers from day 3 to day 7 on both substrates, cells were stained for F-actin protein using rhodamine-phalloidin and for nuclei using sytox green (Magnification 20×; Scale bar: 50 μm)); and FIG. 7E: High magnification confocal image of HUVEC cultured on PFC mat for 7 days (images 1 and 2 represent the combined confocal image, 3 represents only the cell nuclei), in the magnified region the cuboidal shape of endothelial cells is illustrated (cells were stained for F-actin protein using rhodamine-phalloidin and for nuclei using sytox green (magnification 40×; scale bar: 50 μm)).

FIGS. 8A-8D display confocal images illustrating platelet adhesion on different substrates. Platelet rich plasma (PRP) was plated with 1.08×10′ platelets/dish in 48-well plate. The images were acquired after 15 min of incubation on various substrate surfaces at 37° C. FIG. 8A: PFC mat, FIG. 8B: collagen mat, FIG. 8C: culture dish, FIG. 8D: collagen gel. There are more adhered platelets and formation of microthrombi on the collagen gel and collagen mat as compared to the culture dish and the PFC mat. Platelets were visualized by rhodamine-phalloidin staining for F-actin protein (magnification 40×; Scale bar: 50 μm).

FIG. 9 illustrates numbers of adhered platelets on different substrates. Adherent platelets were counted from representative photomicrographs taken from culture dish, PFC mat, collagen mat, and collagen gel after 15 minute incubation at 37° C. Data were expressed as means±SEM (n=3). Bars having a single superscript letter are not significantly different, whereas bars having two different superscript letters are significantly different (p<0.05).

FIGS. 10A-10F display scanning electron micrographs: FIGS. 10A and 10D: collagen gel, FIGS. 10B and 10E collagen mat, FIGS. 10C and 10F: PFC mat after 15 minutes of incubation with PRP. The images demonstrate the presence of a single layer of platelets on the PFC mat and minor platelet activation in comparison to activated platelets on the collagen mat and collagen gel (Magnification: 1500× for FIGS. 10A, 10B, and 10C, scale bar: 10 μm; 5000× for FIGS. 10D, 10E, and 10F, scale bar: 1 μm).

FIGS. 11A-11D display confocal images illustrating the association and interaction of platelets with HUVECs on different substrate. PRP were plated at 1.08×10⁷ platelets/dish in a 48-well plate. The images were acquired after 15 minutes of incubation of platelets on various substrate surfaces at 37° C. FIG. 11A: PFC mat, FIG. 11B: collagen mat, FIG. 11C: culture dish, FIG. 11D: collagen gel. Formation of microthrombi on the collagen gel and increased size of platelet aggregates was observed on the collagen gel and collagen mat as compared to the culture dish and PFC mat. F-actin of cells and platelets were stained using rhodamine-phalloidin and nuclei of cells were stained using sytox green (magnification 40×; scale bar: 50 μm).

FIG. 12 illustrates platelet interaction with cells cultured on different substrates. Numbers of adhered platelets were counted from representative photomicrographs taken from culture dish, PFC mat, collagen mat, and collagen gel substrates after 15 minute incubation at 37° C. White bars (□) indicate areas of the material without endothelial cells, whereas grey bars (□) indicate platelet counts for areas with cells. Data are expressed as mean±standard error of the mean (n=3).

FIG. 13A provides light micrographs (200×) of 3T3 fibroblasts cultured on sparse PFC fibers (1,3) and tissue culture plastic (TCP) (2,4) with (3,4) and without (1,2) decellularization with 3% Triton X-100. PFC without cells or decellularization (5). Slides stained with Crystal Violet. FIG. 13B provides light micrographs of 3T3 fibroblasts cultured on dense PFC mats (1), 3T3 fibroblasts cultured on dense PFC mats and decellularized with Triton X-100 (2), and dense PFC mats without cells cultured (3). Slides stained with H&E.

FIG. 14A provides results of ELISA assays of comparison of adsorbed and conjugated syndecan on PFC scaffolds. FIG. 14B shows the loading capacity of SD4 on PFC scaffolds. FIG. 14C shows a comparison of SDF-1a immoblization on scaffolds with and without SD4. FIG. 14D shows the loading capacity of SDF-1α on SD4.

FIG. 15 shows free amine group content, measured as absorbance values, of crosslinked PFC. * p<0.05 (compared to 10% glutaraldehyde). n=3.

### DETAILED DESCRIPTION OF THE INVENTION

Creating a functional heart valve graft that lasts a lifetime is one of the main objectives of cardiovascular tissue engineering (Mirensky et al. (2008) Pediatr. Res. 63(5):559-568; Mol, A. (2004) J. Heart Valve Dis. 13(2): 272-280; Yacoub et al. (2004) Circulation 109(9):1064-1072; Yacoub et al. (2005) Nat. Clin. Pract. Cardiovasc. Med. 2(2): 60-61). While conventional collagen-based heart valves have been used for many years, they eventually fail because of inadequate cell infiltration and insufficient replacement of graft material by tissue remodelling (Simon et al. (2003) Eur. J. Cardio-Thoracic Surg. 23(6):1002-1006). To overcome graft failure due to inadequate tissue remodelling and growth, an improved graft material was created by incorporating collagen protein with mechanically robust silk fibroin, and a hemocompatible synthetic elastomer as well as extracellular matrix. In a particular embodiment, the hemocompatible synthetic elastomer is a polyglycerol derivative ester comprising a polycarboxylic acid. In a particular embodiment, the polyglycerol derivative ester is poly (glycerol sebacate) (PGS). The present invention demonstrates that electrospun grafts created from composites of collagen, particularly type I collagen, silk fibroin, and PGS are stable, less thrombogenic and easier to fabricate than the conventionally used collagen-based grafts for aortic heart valve replacement. Specifically, a graft material that mimics the composition, structure and mechanical properties of native tissue and promotes the formation of an intact nonthrombogenic endothelial cell layer was fabricated. More specifically, the graft material mimics the composition, structure and mechanical properties of native heart valve tissue. In the present invention, an improved heart valve material composed of a collagen, a fibroin, a hemocompatible synthetic elastomer (e.g., PGS), and ECM and/or a heparan sulphate glycoprotein was developed comprising properties similar to those reported for native heart valves. Indeed, the homogeneity of blended composites is demonstrated using Fourier transform spectroscopy, tensile stress, strain, and elastic modulus of the electrospun mats were measured by an Instron mechanical tester.

Functional tests of PFC mats demonstrate a slow degradation rate as compared to other collagen-based grafts. Cells adhere to and proliferate on PFC mats. Endothelial cells are observed to form tight junctions on the material. Several studies with isolated platelets demonstrate that PFC mats are less thrombogenic in comparison to collagen hydrogels and structurally similar to electrospun collagen mats. In summary, the present invention demonstrates that PFC mats provide strong, slowly degradable, and nonthrombogenic grafts that promote cell adhesion and growth. Accordingly, the PFC mats of the instant invention may be used as functional and durable replacement biomaterials.

The present invention provides a resorbable graft (e.g., heart valve graft) material which imitates the native vascular tissue (e.g., aortic valve tissue) based on protein compositions, elastic modulus, stress, and strain. The graft materials were tested for degradation, endothelium formation and platelet adhesion. In a particular embodiment, the PFC mats are conditioned with ECM which facilitates cell proliferation to generate a healthy tissue (e.g., heart valve) capable of growing and lasting a life-time. In a particular embodiment, the PFC mats further comprise a heparan sulphate glycoprotein (e.g., via crosslinking).

The standard criteria for creating grafts are to mimic the compositional and structural characteristics of native tissue. These can be summarized as a favorable surface for cell attachment, high porosity and interconnected network for nutrient transport and cell signalling, and mechanical strength for performing valvular function under physiological stress. Moreover, a consideration of cell proliferation, cell-cell/ECM interactions, and cell function such as the production of non-thrombogenic glycocalyx is essential.

Traditional tissue grafts demonstrate limited cell growth and ECM remodelling (Sacks et al. (2009) Ann. Rev. Biomed. Engr. 11(1):289-313). Therefore, creating grafts which incorporate active cell binding sites, slow degradability, and maintenance of structural and mechanical integrity during the healing process are desperately needed.

In order to create a graft that meets the aforementioned criteria, a strategy of incorporating natural proteins with an elastomer was implemented. Collagen, fibroin and PGS as well as ECM and/or heparan sulphate glycoprotein possess key characteristics useful in constructing a graft material for potential use as a tissue replacement.

In accordance with the instant invention, nonthrombogenic compositions comprising collagen, fibroin, and a hemocompatible synthetic elastomer are provided. In a particular embodiment, the nonthrombogenic composition is electrospun (e.g., comprises electrospun fibers). In a particular embodiment, the nonthrombogenic compositions of the instant invention comprise fibers having a diameter of about 600 to about 5000 nm, about 2500 to about 3500 nm, or about 2900 to about 3000 nm. In a particular embodiment, the nonthrombogenic composition further comprises extracellular matrix (ECM). For example, the nonthrombogenic composition is conditioned with cellular extracellular matrix. In a particular embodiment, nonthrombogenic composition further comprises a heparan sulphate glycoprotein (e.g., a syndecan such as syndecan 4). For example, the heparan sulphate glycoprotein is covalently linked (e.g., crosslinked) to the nonthrombogenic composition. The nonthrombogenic compositions of the instant invention may comprise both a heparan sulphate glycoprotein and extracellular matrix. In a particular embodiment, the nonthrombogenic compositions of the instant invention further comprises electronegative carbohydrates (such as glycosaminoglycans), growth factors, and/or specific cell signalling molecules. In a particular embodiment, the nonthrombogenic composition has a greater thermal transition temperature as compared to the hemocompatible synthetic elastomer alone.

The nonthrombogenic composition of the instant invention may have the following characteristics. In a particular embodiment, the collagen of the nonthrombogenic compositions is type I collagen. In a particular embodiment, the fibroin of the nonthrombogenic compositions is silk fibroin. In a particular embodiment, the hemocompatible synthetic elastomer of the nonthrombogenic compositions is a polyglycerol derivative ester comprising a polycarboxylic acid. In a particular embodiment, the polyglycerol derivative is poly (glycerol sebacate) (PGS). In a particular embodiment, the weight ratio for the collagen per total composition weight is about 10% to about 90%, about 10% to about 80%, about 25% to about 65%, about 30% to about 60%, about 40% to about 50%, about 43% to about 47%, or about 45%. In a particular embodiment, the weight ratio for the fibroin per total composition weight is about 10% to 90%, about 10% to about 80%, about 25% to about 65%, about 30% to about 60%, about 40% to about 50%, about 43% to about 47%, or about 45%. In a particular embodiment, the weight ratio for the hemocompatible synthetic elastomer (e.g., a poly glycerol derivative ester comprising a polycarboxylic acid or poly (glycerol sebacate)) per total composition weight is about 1% to about 80%, about 10% to about 80%, about 10% to 20%, about 1% to about 20%, about 5% to about 15%, about 8% to about 12%, or about 10%. In a particular embodiment, the weight ratio for the collagen per total composition weight is about 40% to about 50%, the weight ratio for the fibroin per total composition weight is about 40% to about 50%, and the weight ratio for the hemocompatible synthetic elastomer (e.g., a poly glycerol derivative ester comprising a polycarboxylic acid or poly (glycerol sebacate)) per total composition weight is about 5% to about 15%. In a particular embodiment, the weight ratio for the collagen per total composition weight is about 43% to about 47%, the weight ratio for the fibroin per total composition weight is about 43% to about 47%, and the weight ratio for the hemocompatible synthetic elastomer (e.g., a poly glycerol derivative ester comprising a polycarboxylic acid or poly (glycerol sebacate)) per total composition weight is about 8% to about 12%. In a particular embodiment, the weight ratio for the collagen per total composition weight is about 45%, the weight ratio for the fibroin per total composition weight is about 45%, and the weight ratio for the hemocompatible synthetic elastomer (e.g., a poly glycerol derivative ester comprising a polycarboxylic acid or poly (glycerol sebacate)) per total composition weight is about 10%. In a particular embodiment, the “total composition weight” used hereinabove is in reference to the weight of the nonthrombogenic composition comprising collagen, fibroin, and the hemocompatible synthetic elastomer (e.g., a poly glycerol derivative ester comprising a polycarboxylic acid or poly (glycerol sebacate)) (e.g., to the exclusion of other components such as heparin sulfate glycoprotein, ECM, etc.). In a particular embodiment, the nonthrombogenic composition of the instant invention comprises collagen, fibroin, and the hemocompatible synthetic elastomer (e.g., a poly glycerol derivative ester comprising a polycarboxylic acid or poly (glycerol sebacate)) at a weight ratio of about 4 to about 5:about 4 to about 5:about 0.5 to about 1.5; about 4.3 to about 4.7:about 4.3 to about 4.7:about 0.8 to about 1.2; or about 4.5:about 4.5:about 1.0.

Articles of manufacture comprising the nonthrombogenic composition are encompassed by the instant invention. In a particular embodiment, the present invention encompasses a porous electrospun graft material comprising the nonthrombogenic composition (e.g., as described herein). The graft material is readily configured to function as a tissue replacement. The porosity of the graft material may have variable degrees of porosity (e.g., to allow different degrees of infiltration). In a particular embodiment, the graft material a sheet material. In one embodiment, the tissue to be replaced is heart or vascular tissue. In another embodiment, the graft is a heart valve (e.g., an aortic heart valve). In a particular embodiment, the graft is a stent. In a particular embodiment, the graft is a conduit (e.g., with a diameter less than about 20 mm, less than about 15 mm, less than about 10 mm, or less than about 6 mm). In a particular embodiment, the graft is an arterial or venous conduit. in a particular embodiment, the graft is for coronary artery bypass surgery.

In a particular embodiment, the graft degrades at a rate slower than collagen. In a particular embodiment, the graft is less thrombogenic than collagen. In a particular embodiment, the graft is structurally and mechanically similar to native tissue. In a particular embodiment, the graft is configured to promote cell adherence and proliferation. In a particular embodiment, the graft is configured to promote the formation of an intact nonthrombogenic endothelial cell layer. In a particular embodiment, the graft is resorbable. In a particular embodiment, the graft is configured to facilitate cell proliferation and ECM remodeling. In a particular embodiment, the graft comprises a porous network of interconnected fibers effective to facilitate nutrient transport and cell signaling. In a particular embodiment, the graft is configured to facilitate the formation of tight cell-cell junctions between cells proliferating in or on the graft. In a particular embodiment, the graft is sufficiently porous to facilitate cell adhesion, nutrient transport and signal transmission.

In a particular embodiment, the graft comprises a fibrous structure. In a particular embodiment, the graft comprises a porous network of interconnected fibers. For example, the porous network may be about 100 to about 300 μm thick. In a particular embodiment, the graft or nonthrombogenic composition of the instant invention has a porosity of about 1% to about 99%, about 10% to about 95%, about 50% to about 90%, or about 67% to 87%.

In a particular embodiment, the graft has an elastic modulus, stress value, and strain value substantially similar to tissue to be replaced (e.g., native heart valve, artery, etc.). In a particular embodiment, the graft has an elastic modulus from about 2 to about 5 Mpa, about 2.3 to about 5.0 Mpa, or about 4.00 to about 4.25 Mpa. In a particular embodiment, the graft has a stress value of about 0.6 to about 2.0 Mpa, about 0.6 to about 1.5 Mpa, or about 1.3 to 1.5 Mpa. In a particular embodiment, the graft has a strain value of about 0.2 to about 0.7 mm/mm or about 0.4 to about 0.5 mm/mm.

In an additional aspect, the present invention encompasses a method of preparing a graft (e.g., for heart valve or arterial replacement), where the method comprises the steps of: (a) preparing (e.g., electrospinning) a nonthrombogenic composition of the instant invention, thereby generating an interconnected nanofiber matrix; (b) conditioning the interconnected nanofiber matrix with ECM; and (c) collecting the interconnected nanofiber matrix to produce the graft (e.g., for heart valve or vascular replacement). In a particular embodiment, step (a) is performed in a mold to produce an interconnected nanofiber matrix having the desired shape (e.g., heart valve or arterial graft). In a particular embodiment, the method further comprises preparing an electrospinning solution comprising collagen, fibroin and a hemocompatible synthetic elastomer. In a particular embodiment, the method further comprises crosslinking the nanofiber matrix/graft (e.g., to crosslink collagen fibers; e.g., by glutaraldehyde). In a particular embodiment, the method further comprises heat treating the nanofiber matrix/graft (e.g., at about 120° C. for about 48 hours). In a particular embodiment, the nanofiber matrix comprises a heparan sulphate glycoprotein (e.g., heparan sulphate syndecan, particularly syndecan-4).

In a particular embodiment, step (b) comprises culturing the interconnected nanofiber matrix with cells which produce extracellular matrix (e.g., fibroblast or human aortic smooth muscle cells (HASMCs)). In a particular embodiment, the extracellular matrix producing cell is a fibroblast. In a particular embodiment, the interconnected nanofiber matrix is cultured with the cells which produce extracellular matrix for about 1 to about 30 days, particularly about 3 to about 20 days, about 5 to about 15 days, or about 10 days. In a particular embodiment, the method further comprises lysing the cultured cells by delivering a lysing agent to the cells cultured with the interconnected nanofiber matrix (e.g., before or after (c)). Examples of lysing agents include, without limitation, detergents and chaotropic agents. Chaotropic agents include, but are not limited to, guanidinium hydrochloride, guanidinium thiocyanate, guanidinium isothiocyanate and sodium iodide. Chaotropic salts can also be used which comprise a chaotropic anion selected form the group consisting of trichloroacetate, perchlorate and trifluoroacetate. Detergents can be ionic and/or non-ionic detergents. Non-ionic detergent include, without limitation, Triton™ X100, polysorbate (e.g., Tween® 20), Brij35 or NP-40.

In a particular embodiment, methods of increasing the porosity of a graft of the instant invention are provided. The grafts of the instant invention may have increased porosity by adding a polymer, particularly a hydrophilic polymer such as poly(ethylene oxide) (PEO), to the nanofiber matrix (e.g., the electrospun nonthrombogenic composition). The nanofiber matrix is then crosslinked with a crosslinker (e.g., formaldehyde, paraformaldehyde, acetaldehyde, glutaraldehyde, etc., particularly glutaraldehyde) and the polymer (e.g., PEO) is then leached out of the nanofiber matrix (e.g., with alcohol).

Methods of adding a heparan sulphate glycoprotein (e.g., heparan sulphate syndecan) to the compositions/grafts of the instant invention are also provided. In a particular embodiment, heparan sulphate glycoprotein is covalently attached to a component of the nonthrombogenic composition (e.g., PFC mat) by crosslinking. In a particular embodiment, the nonthrombogenic composition (e.g., PFC mat) are crosslinked (e.g., by formaldehyde, paraformaldehyde, acetaldehyde, glutaraldehyde, etc., particularly glutaraldehyde; particularly at an amount less than about 5%, less than about 1.5%, or about 0.5%) prior to heparan sulphate glycoprotein linkage. In a particular embodiment, the method further comprises contacting the heparan sulphate glycoprotein modified nonthrombogenic composition with stromal cell-derived factor 1 (SDF-1α).

Type I Collagen.

Specifically, among the natural proteins, collagen is the most abundant load-bearing component of aortic valve cusp, while elastin imparts flexibility to soft tissue. The fundamental unit of the fibrillar collagen is the triple helix. The triple helix is made up of 3 polypeptide chains that each are 1000 amino acid long with glycine-X-Y (Gly-X-Y) repeats (Alberts et al. (2002) Molecular Biology of the Cell, Garland Science Taylor & Francis Group; Malafaya et al. (2007). Adv. Drug Del. Rev. 59(4-5): 207-233). The amino acid sequences, such as RGD (Arg-Gly-Asp), DGEA (Asp-Gly-Glu-Ala) or GFOGER (Gly-Phe-Hyp-Gly-Glu-Arg) in type I collagen motifs specially binds to α2β1 integrin to regulate cell adhesion (Gu et al. (2010) J. Biomed. Matr. Res., Part A 93A(4):1620-1630; Shekaran et al. (2011) Biochim. et Biophys. Acta 1810(3):350-360). As the major structural protein, type I collagen absorbs most of the stress during the closing of the aortic valve in diastole when the ventricle is filled with blood.

Poly (glycerol sebacate) (PGS).

The polymer poly (glycerol-sebacate) (PGS) mimics the mechanical behavior of the ECM protein, elastin (Pomerantseva et al. (2009) J. Biomed. Mater. Res., Part A 91A(4):1038-1047). It has low elastic modulus and large elongation capacity that is similar to elastin in valvular ECM (Alberts et al. (2002) Molecular Biology of the Cell, Garland Science Taylor & Francis Group; Sant et al. (2010) Fabrication and characterization of tough elastomeric fibrous scaffolds for tissue engineering applications. Engr. Med. Biol. Soc. (EMBC), 2010 Ann. Intl. Conf. of the IEEE; Sant et al. (2011) J. Tissue Engr. Regen. Med. 5(4): 283-291). PGS has been reported to promote synthesis of mature and organized elastin, as well as having a superior hemocompatibility over other synthetic polymers such as poly (1-lactide-co-glycolide) (PLGA) (Motlagh et al. (2006) Biomaterials 27(24):4315-4324.; Lee et al. (2011) Proc. Natl. Acad. Sci. 108(7): 2705-2710). PGS was also reported to be biocompatible in vivo and in vitro. Endothelial cells and fibroblasts were viable when cultured with PGS (Wang et al. (2002) Nat. Biotech. 20(6): 602-606; Yi et al. (2008) Macromol. Biosci. 8(9):803-806). Minimal inflammatory response and no fibrous collagen capsules were observed for PGS (Wang et al. (2002) Nat. Biotech. 20(6): 602-606). The elastic property and biocompatibility of PGS make it a potent biomaterial for cardiovascular tissue grafts.

Silk Fibroin.

To improve the strength of graft material and incorporate slow degradability, silk fibroin was selected (Horan et al. (2005) Biomaterials 26(17):3385-3393). The adjacent -(Ala-Gly)- repeated sequence forms polypeptide chains with molecular weights of 390 kDa (heavy chain) and 25 kDa (light chain). Serving as the structural and major protein in the silk, fibroin protein polypeptide chains have interchain hydrogen bonds that contribute to the special highly crystalline (3-sheet conformation. Interchain hydrogen bonds in silk fibroin protein assemble the polypeptide chains into the highly crystalline (3-sheet conformation which imparts a slow degradation rate. Degradation is defined as the breakdown of the materials and leads to changes in physical properties. The degradation rate of silk fibroin is controllable and may be modified to last from hours to years (Rockwood et al. (2011) Nat. Protocols 6(10):1612-1631). By incorporating these properties of silk fibroin, the resulting graft material provides sufficient mechanical support and performs the physiological function of valve tissue. Extracellular matrix remodelling takes up to 20-weeks. The slow degradation rate allows maintenance of a durable functional graft before cell infiltration and growth can take place (Horan et al. (2005) Biomaterials 26(17):3385-3393). This is particularly important for heart valve and vascular grafts due to their special requirements in functionality and durability.

Fibroin possesses greater tensile property than collagen but possesses little elasticity. Collagen has a multitude of cell binding motifs. PGS, for its part, provides elasticity. The designed material has better mechanical properties than reported biomaterials, decellularized valves, or decellularized heart muscle that have been used for valve replacement. The invention described herein was developed to improve the tensile and durability properties of biomaterials.

With regard to cell adhesion, it was theorized that adding ECM components onto the PFC material would strengthen the material and deposit molecules that facilitate cellular adhesion and proliferation. As seen hereinbelow, the ECM components improved cellular adhesion.

Based on the greater binding and proliferation of cells, it was also expected that there would be a similar or significantly greater adhesion of platelets to the PFC compound to collagen. However, quite surprisingly, the opposite effect was observed, i.e., reduced numbers of platelets adherent to PFC compared to physically similar collagen nanofibers.

Describing the present invention in further detail, in certain embodiments, materials fabricated with varying weight ratios of collagen, fibroin, and PGS had elastic moduli between 2.3-5.0 Mpa; tensile stress ranging from 0.6 to 1.5 Mpa; and strain values between approximately 20%-70%, which were similar to reports for native heart valves. Mechanical and suture retention tests (a highest 0.32N pull-out force at the single-loop suture site) indicated electrospun mats with 4.5:4.5:1 collagen, fibroin, and PGS weight ratio (PFC fiber mats) were most similar to native heart valves. Over a 30 week period in vitro degradation of PFC mats was only 0.01% per week with no significant change in fiber diameter. Endothelial cells adhered to and proliferated on PFC mats, and formed tight cell-cell junctions. Platelets adhesion studies surprisingly showed 2.2-2.9 fold less platelet adhesion compared to collagen hydrogels and electrospun collagen mats, respectively.

Electrospinning Fabrication.

Electrospinning is a fabrication technique that is applied to rapidly create a graft. Moreover, electrospinning fabrication is able to provide nanofiber porous networks in the form of an ultra thin sheet for the application of making grafts (e.g., heart valve or vascular grafts). In other embodiments, a mold may be used to create electrospun grafts or other electrospun structures of different shapes in order to, for example, mimic different valve structures, prepare stents, or fabricate other biologically relevant structures.

Indeed, in addition to the composition, the structure of the material is another important design consideration. Large surface area and sufficient porosity allow cell adhesion, nutrient transport, and signal transmission to enhance cell response for tissue remodelling. Production of fibers having pre-determined diameters and alignments can be achieved by controlling electrospinning parameters such as solution viscosity, voltage, environmental humidity, and collector orientation (Sell et al. (2010) Polymers 2(4):522-553). Polymer composites are dissolved in appropriate solvent such as 1, 1, 1, 3, 3, 3-hexafluoro-2-propanol (HFP) to form the electrospinning solution. The electrospinning solution usually is loaded into a syringe that is placed on a pump to inject the solution at a constant rate. A high voltage power source is connected to a conductive syringe tip. A conductive collector is grounded on the opposite side to the syringe to create an electric field. Polymer solution at the syringe tip forms a small droplet called a Taylor cone. When the electrostatic force overcomes the surface tension of the polymer solution, jets of polymer solution travel toward the collector and form an interconnected fiber matrix. In practicing the present invention, collagen, fibroin and polyglycerol polymer are electrospun into an interconnected nanofiber matrix graft material.

Silk Fibroin Extraction.

Silk fibroin protein was extracted according to the published procedure with modifications (Rockwood et al. (2011) Nat. Protocols 6(10):1612-1631). Raw silk was boiled in 2 L of 0.02 M Na₂CO₃ at 100° C. for 30 minutes, rinsed twice with DDH₂O, squeezed, and air dried. Fibroin was then dissolved in 5.0M CaCl₂, and centrifuged at 2000 g to remove precipitate and floating contaminants. The fibroin solution was dialyzed and lyophilized to obtain powder for electrospinning. The identity and purity of fibroin was confirmed by amino acid assay analysis (Lombardi et al. (1990) J. Arachnol. 18(3): 297-306; Schroeder et al. (1955) J. Amer. Chem. Soc. 77(14):3908-3913; Zhou et al. (2001) Proteins: Struct. Func. Bioinf. 44(2):119-122). The amino acid composition of extracted fibroin was identified as conforming to reported values (Schroeder et al. (1955) J. Amer. Chem. Soc. 77(14):3908-3913). Major amino acids glycine, alanine, and serine comprised 82% of the total amino acids in contrast to sericin that has 11% glycine, 70% alanine, and 33% serine (Swiss-Prot accession number: P07856).

The molecular weight of fibroin was determined using sodium dodecyl sulphate-polyacrylamide gel electrophoresis (SDS-PAGE) followed by staining with Coomassie blue (Horan et al. (2005) Biomaterials 26(17):3385-3393). The heavy (390 kDa) and light (25 kDa) chains of fibroin were observed, while a 119 kDa band indicative of serine was absent.

Synthesis of PGS Prepolymer.

PGS prepolymer can be synthesized from glycerol and sebacic acid which have been approved by FDA for medical applications (Wang et al. (2002) Nat. Biotech. 20(6): 602-606). The synthesis procedure followed the reported standard method with modification (Wang et al. (2002) Nat. Biotech. 20(6): 602-606; Pomerantseva et al. (2009) J. Biomed. Mater. Res., Part A 91A(4):1038-1047). Briefly, sebacic acid was heated at 180° C. on an oil bath with nitrogen flow across the reaction flask for 10-20 minutes until all melted. An equimolar amount of warmed glycerol (60° C.) was added. Then the pressure was decreased by attaching to a vacuum (General Medical, Richmond, Va.) and the temperature was kept at 150° C. for 4 hours to obtain PGS prepolymer in the form of a viscous amber color solution.

Because the PGS prepolymer was in a viscous aqueous form, it usually requires thermal curing at 120° C. for 48 hours to form the solidified PGS elastomer (Yi et al. (2008) Macromol. Biosci. 8(9):803-806). In the present invention, PGS prepolymer was used in making the polymer and protein solution blends for electrospinning.

Production and Characterization of the Electrospun Mats.

Solutions for production of nanofiber mats were prepared using type I collagen (Collagen type I from calf skin was purchased commercially (Elastin Products Corp, MO)), silk fibroin, and PGS, synthesized as described above, at different weight ratios of 9:0:1, 8:1:1, 4.5:4.5:1, 1:1:8, and 0:9:1, which were dissolved in HFP respectively. The syringe loaded with the solution was fixed on a Baxter infusion pump (Model AS50) to eject the polymer solution at a rate of 3 ml/hour. A 35 kilovolt high voltage (Gamma High Voltage Research, Ormond Beach, Fla.) was applied, and a distance of 20 cm was provided between the metal collector plate (12 cm by 12 cm) and syringe tip. The electrospun mats were then placed in a 120° C. oven for 48 hours to thermally crosslink PGS (Yi et al. (2008) Macromol. Biosci. 8(9):803-806). Proteins were chemically crosslinked with glutaraldehyde vapor for 24 hours (Sung et al. (2000) Biomaterials 21(13):1353-1362).

Morphology of Electrospun Mats.

Images of the electrospun mats were obtained using scanning electron microscopy (JEOL JSM-6330F) and used to measure fiber diameters. Using NIH Image J software, sixteen random measurements of fiber diameters from each mat were obtained.

Characterization of Chemical Functional Groups and Physical Properties of Electrospun Mat.

Chemical functional groups were detected using a Perkin-Elmer FT-IR spectrophotometer to identify the polymer and protein structures. The thermal transition temperatures were detected following differential scanning calorimetry (DSC) (TA instruments, New Castle, Del.). Thermal transition curves of electrospun mats at various composite ratios were obtained from −60° C. to 300° C. at an increment rate of 20° C./min. All mats were randomly sampled in triplicate.

Thermal Transition Analysis.

The transition in physical state due to temperature change is important to implantable grafts. It is necessary to obtain a comprehensive thermal transition profile of electrospun mats with temperatures ranging from below storage temperature to above autoclave temperature. Phase changes of the materials are associated with exothermic (release heat) and endothermic (absorb heat) reactions. These thermodynamic changes can be detected by differential scanning calorimetry (DSC). Samples were prepared using a 3 mm biopsy punch and placed inside of standard Tzero aluminum pan/lid pairs (TA instruments, New Castle, Del.). An empty pan of the same materials was used as a reference. In each group, three samples were prepared and tested. The aluminum pan/lid pairs were weighed before and after loading the samples to calculate the sample weight. Thermal stabilization with no endo/exothermic events was achieved before the sample reached −60° C. (Simone et al. (2009) Biomacromolecules 10(6):1324-1330). To obtain the complete profile of thermal transitions of the various composite ratios of electrospun mats, the DSC was run with the following steps: (1) Equilibrate to −70° C. Ramp 20° C./min to 300° C. (heat); (2) Mark end of cycle 0; (3) Ramp 20° C./min to −70° C. (cool); (4) Mark end of cycle 1; (5) Ramp 20° C./min to 20° C. (heat); (6) Mark end of cycle 2.

The obtained thermal transitions depicted by heat flow over temperature were individually plotted and analyzed. By analyzing the thermal transition curves measured by DSC, changes in protein conformations and assemblies of the peptide chains can be studied. The glass transition may be seen as a step endotherm illustrating the state transition of the material from crystalline to amorphous phase. The melting point seen at the peak of a large endotherm depicted the material transition from solid to fluidic state of the material.

For collagen, temperature induced denaturation disrupts the hydrogen bonding between the polypeptide chains, and disassembles the helical structures to form a random coil. For highly crystalline fibroin, the temperature induced phase transformation is important to understand the protein conformation. These changes can be reflected by endotherms in the thermal transition curve.

Porosity Measurements.

Following hydration in 5 ml DDH₂O on a mechanical shaker for 30 minutes at 22° C., mat samples were blotted dry and the weights were measured again (Kim et al. (2003) Biomaterials 24(27):4977-4985). The density of type I collagen (1.40 g/cm³), silk fibroin (1.31 g/cm³), and PGS (1.13 g/cm³) and the weight ratios were used to calculate the densities of the composites (Pomerantseva et al. (2009) J. Biomed. Mater. Res., Part A 91A(4):1038-1047; De Cupere et al. (2003) Langmuir 19(17):6957-6967.; Minoura et al. (1990) Polymer 31(2):265-269). The volume of each electrospun mat sample was obtained by dividing the weight by the density of the mat, and then the porosity was calculated using the following equation

$$ɛ = \frac{{V}_{liq}}{{V}_{liq} + {V}_{MAT}}$$

(Soliman et al. (2011) J. Biomed. Mater. Res., Part A 96A(3): 566-574). Where V_{iiq} is the volume of the intruded water and V_{MAT} is the volume of the electrospun mat (Kim et al. (2003) Biomaterials 24(27):4977-4985).

Mechanical Tensile Testing.

The standard dumbell stamp (ASTM D638-IV cutting die, Pioneer-Dietecs Corporation, Norwood, Mass.) was used to prepare samples of each electrospun composite. An Instron 5500R mechanical tester (Instron Corporation, Norwood, Mass.) with a 500N load cell and Blue Hill software was used to perform uniaxial tensile tests of the mats after hydration in 100 ml DDH₂O for 10 minutes at an elongation rate of 10 mm/min. Three measurements from each type of electrospun mat were used to calculate elastic modulus, stress, and strain values.

Modified Suture Retention Testing.

A suture retention test performed in the manufacture of heart valve is capable of modification from a published protocol (Trowbridge et al. (1989) J. Biomed. Engr. 11(4):311-314). Five samples of each mat measuring 2 cm in length and 0.5 cm in width were prepared. The monofilament prolene suture (3-0 monofilament; Ethicon, Somerville, N.J.) was placed 0.5 cm from the edge to form a single loop. Samples were loaded onto the BOSE-Electroforce mechanical tester, and stretched at a rate of 10 mm/minute until the suture completely ripped the material.

Material Degradation.

To assess degradation, 5 random samples of PFC mats (at 4.5:4.5:1 weight ratio of collagen:fibroin:PGS) were incubated at 37° C. in 10 ml phosphate buffered saline (PBS) containing 0.1% sodium azide. Samples from 3 batches were tested in separate experiments. The weights of samples were measured every week over a 30 week period. The percentages of weight loss was calculated as the ratio of mass change after degradation to the original mass of the scaffold according to the formula Weight loss (%)=W1/Wix 100%. (Wi: =initial weight of the electrospun fiber mats and W1=weight loss of the same fiber mats after exposure in degradation solution) (Okhawilai, M. (2010) Intl. J. Biol. Macromol. 46(5):544-550; Liu et al. (2010) J. Biomed. Mater. Res., Part A 95A(1):276-282). At each time point, one sample was lyophilized and processed for SEM and used to evaluate fiber morphology.

Cell Adhesion and Proliferation.

Heart valve and vascular grafts are directly in contact with blood. Whole blood is composed of red blood cells, white blood cells, platelets, and plasma proteins. In the body, platelets adhere to sites of vascular lesions and participate in one of the initial events of thrombosis (blood clot). A monolayer of endothelial cells which lines both surfaces of valvular tissue provides further mechanical strength to valvular ECM and a protective layer to prevent thrombosis. The endothelial cells prevent platelets from coming into contact with subendothelial collagen which leads to platelet activation and thrombosis (Zhu et al. (2010) Blood 2116(2023):5050-2019). Therefore, a graft material that promotes the formation of an endothelial cell monolayer on the graft in a continuous manner is important for ensuring the functionality and hemocompatibility of the implant.

Hemocompatibility.

The interaction of platelets with the electrospun mats of the present invention was observed. This is because platelet activation plays an important role in thrombosis which will directly affect the hemocompatibility and success of a heart valve or vascular graft. An amino acid sequence -Arg-Gly-Asp (RGD) on proteins such as collagen can activate platelet aggregation by inducing high binding affinity of the platelets. The mechanism proceeds through binding of the glycoprotein GPIb and GPIIb/IIIa receptor on the platelet surface to the proteolytic factor von Willebrand factor, which recognizes and binds to the binding domains in thrombogenic materials, such as collagen. Such signalling will changes the conformation of integrin on the platelet surface and leads to thrombus formation (Zhu et al. (2010) Blood 2116(2023):5050-2019; Mendelboum Raviv et al. (2012) Thrombosis Res. 129:e29-e35). To test the hemocompatibility of graft materials embodying the invention, platelet activation was studied by the number of adhered platelets and their morphologies on various substrates.

Cell Culture Study.

To test cell-material interaction, PFC mats were sterilized with ethanol followed by ultra-violet (UV) exposure to each side for 1 hour. In order to mimic in the in vivo condition where plasma proteins coated implanted material, fibronectin (Sigma, St Louis, Mo.) was used at a concentration of 100 μg/ml to coat mats and culture dishes. HUVECs (ATCC: CRL-1730) were seeded on the substrates in a 16 well culture plate at a cell density of 50,000 cells/well. On day 3 and day 7, rhodamine-phalloidin and sytox green dyes (Invitrogen, Eugene, Oreg.) were used, respectively, to visualize F-actin and nuclei using a Zeiss confocal microscope. Image J software (NIH) was used to count the cell numbers from representative confocal imaging micrographs.

Platelet-Material Interaction.

Human whole blood was drawn into a 2.7 ml BD Vancutainer® Coagulation Tube (BD, Franklin Lakes, N.J.) and centrifuged at 800 rpm for 15 min at 25° C. with Harrier 18/80 centrifuge (Sanyo Gallenkamp, Loughborough, UK) to obtain Platelet rich plasma (PRP). In a first experiment, PFC mats, type I collagen mats or hydrogels of rat tail type I collagen (BD Biosciences, Bedford, Mass.) hydrogels were placed in culture dishes and used as substrates for platelet adhesion. PRP is diluted with PBS to 2.16×10⁸ platelets/ml, and 100 μl was applied to the center of the substrates. After 15 minutes materials are washed two times with PBS followed by fixation in 2.5% glutaraldehyde. F-actin, the contractile protein expressed in platelets, was stained with rhodamine-phalloidin to identify platelet interactions with materials. The amount of adherent platelets on various substrates was observed using confocal microscopy and the morphology of platelets were observed using SEM. Platelet adhesion was quantified using a cell counter and Image J software. In a second experiment, HUVECs were cultured on different materials for 24 hours prior to the addition of platelets. The same protocol was used for platelet addition and evaluation of platelet adhesion. Three pictures were taken from randomly selected different areas on the substrates. The numbers of platelets were counted and compared among groups.

Statistical Analysis.

Data is presented as mean±standard error of the mean (SEM) unless otherwise noted. Statistical significance was determined using one-way analysis of variance (ANOVA) following post hoc test for multiple groups when appropriate. A value of p<0.05 was considered statistically significant. StatView 5.0 (SAS Institute, Cary, N.C.) was used to perform the statistical analyses.

Mechanical and physical tests on the PFC mats embodying the present invention demonstrate that the material has superior strength and flexibility compared to primarily collagen rich biomaterials. The β sheet structure of fibroin normally results in a stiff but brittle material. However, blending silk fibroin with collagen and PGS created a novel and surprisingly tough material with elastic properties. A composition of 90% fibrous protein and 10% elastic protein was initially considered optimum based on the composition of native heart valve. While 10% PGS was thought to be sufficient to substitute for elastin based on natural valve composition, collagen and fibroin optimization was necessary. Results showed electrospun mats at 4.5:4.5:1 weight ratio of collagen, fibroin, and PGS was optimum based on comparisons made with native heart valve. In the suture retention tests, PFC mats showed satisfactory suture retention force as compared to fresh porcine heart valve, indicating that PFC mats could be used effectively as surgical implants.

Creating valvular grafts capable of retaining mechanical integrity by resisting cell-mediated mechanical buckling and microstructure failure is a major challenge (Cebotari, S. (2011) Circulation 124(11 suppl):5115-123; Dijkman et al. (2012) Biomaterials 33(18):4545-4554). One approach involves having cells seeded first on synthetic grafts in order to deposit ECM composites. Prior to the use of these ECM “conditioned” grafts, cells were removed to prevent further contraction (Dijkman et al. (2012) Biomaterials 33(18):4545-4554). The present invention utilized a different strategy, which is fundamentally to improve the strength of the graft material by appropriate selection of components so as to prevent cell-mediated buckling. This was accomplished by incorporating silk fibroin with helical collagen and the elastomer PGS to obtain both strength and flexibility (Beun et al. (2011) ACS Nano 6(1):133-140; Billiar et al. (2000) J. Biomech. Engr., 122(4):327-335). The brittleness of silk fibroin was modified by incorporating collagen and the elastic PGS. An interconnected, porous meshwork was fabricated using electrospinning. Silk fibroin protein has unique anti-parallel β sheet structure making the composite stiff (Jiang et al. (2007) Adv. Funct. Mater. 17(13):2229-2237). The FTIR suggested amides slightly shifted from higher wavenumbers to lower wavenumbers as the collagen content decreased and fibroin content increased in the composites. These shifts corresponded to changes in a random coil structure that was present in collagen and the β sheet structure that was characteristic for fibroin as the composite ratio changed (Hu et al. (2006) Macromolecules 39(18):6161-6170; Zoccola et al. (2008) Biomacromolecules 9(10):2819-2825).

PFC mats showed minimal weight loss during a 30 week degradation study and the ultrastructure of the nanofibers showed little if any changes. Notably, the structural integrity was superior to the reported degradation of fully crosslinked collagen or polylactic acid electrospun constructs (Horan et al. (2005) Biomaterials 26(17):3385-3393; Zong et al. (2003) Biomacromolecules 4(2):416-423; Liu et al. (2010) J. Biomed. Mater. Res., Part A 95A(1):276-282; Kim et al. (2003) Biomaterials 24(27):4977-4985). The results of the studies on PFC mats indicate that silk fibroin degrades slower than most of other collagen-containing scaffolds, such as poly caprolactone-collagen scaffolds which completely degraded in 4 weeks (Zhou et al. (2010) Polymer Degrad. Stab. 95(9):1679-1685; Tedder, M. E. (2009) Tissue Engr. Part A 15(6):1257-1268). These studies indicate that PFC mats should be stable at 37° C. following implantation in patients. Indeed, the PFC mats demonstrated an unexpectedly slower rate of degradation as compared to collagen alone.

Cell attachment and growth is considered to be the first step for achieving sufficient tissue remodelling and maturation of implanted heart valves or conduits in vivo (Butcher et al. (2011) Adv. Drug Del. Rev. 63(4-5):242-268). In the present invention HUVEC cells attached and proliferated on PFC mats and produced a monolayer with tight junctions. A high level of cell adhesion and the potential for tight cell-cell interactions translates into improved mechanical strength of the graft material (Edwards et al. (2005) J. Cardiovasc. Magn. Reson. 7(5):835-840).

While not wishing to be restricted to any specific theory of operation, it is believed that 1) individual collagen, fibroin, PGS, and ECM components in the fibrous mats provide binding sites and microenvironment cues for guiding cell adhesion and proliferation (Gu et al. (2010) J. Biomed. Matr. Res., Part A 93A(4):1620-1630; Shekaran et al. (2011) Biochim. Biophys. Acta 1810(3):350-360; Zou et al. (2012) J. Biol. Chem. 287(10):7190-7202); and 2) in contrast to other materials, the PFC mats of the present invention are effective in producing a better quality glycocalyx.

The special structural features of PFC mats could potentially contribute to long term viability of valvular cells. The interconnected nanofiber network had a thickness of 100-300 μm and could efficiently support nutrient, oxygen transport and soluble cell signal transmission. The electrospun fibers of PFC mats mimicked the highly porous matrix structure of valvular ECM which is essential in providing a large surface area for cell attachment and growth.

To improve the functionality of PFC mats, electronegative carbohydrates such as glycosaminoglycans, growth factors or specific cell signalling molecules can be further incorporated to provide mechanical and chemical cues to cells in biomaterials (Jordan et al. (2012) J. Thor. Cardiovasc. Surg. 143(1):201-208.; De Cock, L. J. (2010) Biomacromolecules 11(4): 1002-1008; Yamada et al. (1980) J. Biol. Chem. 255(13):6055-6063; Deng, C. (2011) J. Huazhong Univ. Sci. Tech. Med. Sci. 31(1):88-93). These further functionalized materials may contribute to a higher level of mature valvular tissue formation by attracting and integrating larger amount and more viable native valvular or progenitor cells.

In a particular embodiment, the compositions/mats of the instant invention further comprise a heparan sulphate glycoprotein (e.g., heparan sulphate syndecan). In a particular embodiment, heparan sulphate glycoprotein is covalently attached to a component of the nonthrombogenic composition (e.g., PFC mat). In a particular embodiment, the heparan sulphate glycoprotein of the nonthrombogenic composition is attached/bound to stromal cell-derived factor 1 (SDF-1a). Heparan sulfate proteoglycans are glycoproteins comprising one or more covalently attached heparin sulfate (HS) chains, a type of glycosaminoglycan. Examples of heparan sulphate glycoproteins include, without limitation, syndecans, glycosylphosphatidylinositol-anchored proteoglycans (glypicans), secreted extracellular matrix heparan sulphate glycoproteins (e.g., agrin, perlecan, type XVIII collagen), and the secretory vesicle proteoglycan serglycin. In a particular embodiment, the heparan sulphate glycoprotein is a syndecan (e.g., syndecan-1, syndecan-2, syndecan-3, and/or syndecan-4). In a particular embodiment, the heparan sulphate glycoprotein is syndecan 4.

In a particular embodiment, the heparan sulphate glycoprotein is covalently attached (e.g., via a linker) or crosslinked to the nonthrombogenic composition (e.g., PFC mat). The heparan sulphate glycoprotein may be attached to the nonthrombogenic composition (e.g., PFC mat) at any chemically feasible location. The term “crosslinker” refers to a molecule capable of forming a covalent linkage between compounds. In a particular embodiment, the crosslinker forms a covalent linkage via a carboxyl-to-amine. Crosslinkers are well known in the art. The crosslinker may be a bifunctional, trifunctional, or multifunctional crosslinking reagent. In a particular embodiment, the crosslinker is non-biodegradable or uncleavable under physiological conditions. In a particular embodiment, the crosslinker is a carbodiimide crosslinker. In a particular embodiment, the crosslinker is 1-ethyl-3-(3-dimethylaminopropyl) carbodiimide (EDC) (optionally with n-hydroxysuccinimide (NETS)).

Studies have shown elevated thrombogenic risks associated with decellularized collagen-based grafts (Schopka, S. (2009) J. Biomed. Mater. Res., Part B, App. Biomat. 88(1):130-138). The present invention demonstrates that the mats of the instant invention are more hemocompatible than structurally similar collagen materials of the prior art. Because the PFC mats are directly synthesized and fabricated from proteins and synthetic polymer, they offer an unlimited off-the-shelf alternative supply, and minimal concerns with respect to disease transmission risk, as compared to processed bovine or porcine grafts. The electrospinning fabrication techniques can be used to rapidly produce PFC mats compared to other heart valve graft processing procedures (Sacks et al. (2009) Ann. Rev. Biomed. Engr. 11(1):289-313). As a feasible fabrication technique, electrospinning on molds could be used to recreate the native geometry of certain tissues, such as heart valve tissue. Other uses might include fabrication onto stents for use as a new generation of transcatheter heart valve for use in minimally invasive cardiothoracic surgery (Dijkman et al. (2012) Biomaterials 33(18):4545-4554).

The present invention demonstrates the formulation of a new composite of natural and synthetic material that can be used for tissue replacement, particularly heart valve or vascular replacement. In the present invention, composites of collagen, fibroin, PGS, and ECM were successfully created and fabricated using electrospinning. In the present invention, composites of collagen, fibroin, PGS, and heparan sulphate glycoprotein were successfully created and fabricated using electrospinning. These compositions provide the unique property of viscoelasticity combined with tensile strength. Accordingly, the compositions of the present invention are useful in a variety of wound care dressings and skin covers, in addition to their use as graft materials for tissue replacement.

In a particular embodiment, the electrospun material is a PFC mat with collagen:fibroin:PGS at 4.5:4.5:1 weight ratio. The compositional and structural similarities of PFC mats to native valvular tissues offer cellular binding sites and microenvironment cues for cell adhesion and growth, but the inclusion of ECM improves upon these properties. The interconnected fibrous structure and porosity of PFC mats provides a large surface area and internal space for tissue maturation to occur. Indeed, the porosity of the PFC mats can be altered to allow more less cellular infiltration. Mechanical testing demonstrated the PFC mats had comparable mechanical strength to fresh heart valve tissue, and therefore could withstand physiological blood pressures. Functionality tests showed minimal weight loss and sustained nanofiber structural integrity over a 9-week study of degradation. Organized endothelial monolayers with tight cell-cell functions formed on PFC mats. Reduced platelet adhesion and aggregate size suggests PFC mats are less thrombogenic compared to collagen nanofiber mats and collagen gels.

In summary, the novel PFC mats created in the present invention may be used as durable, biocompatible, and nonthrombogenic grafts. The fabrication process can be further implemented to mimic the geometry of native heart valve in order to enhance the in vivo functionality and performance of the PFC mats.

The following examples describe the invention in further detail. These examples are provided for illustrative purposes only, and should in no way be considered as limiting the invention.

### Example 1

Composite Characterization.

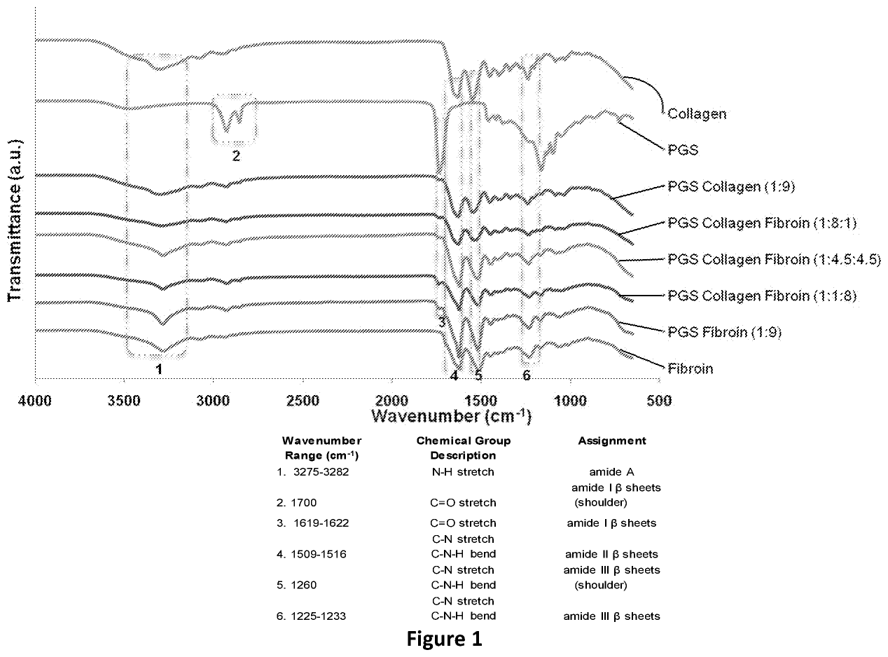

The blending of materials in composite nanofibers is initially demonstrated. For the extracted fibroin, FTIR spectroscopy indicated amide I, II, III groups were in the β sheet conformation based on wavenumbers corresponding to the carbonyl stretchs 1619-1622 cm⁻¹, 1509-1516 cm⁻¹, and 1225-1233 cm⁻¹ respectively. The wavenumber ranging from 3275-3282 cm⁻¹ was indicative of the —N—H stretching vibration shown as a broad peak for amide A. An absorption peak of 1700 cm⁻¹ was assigned to be the C═O stretch in amide I β sheets, and 1225-1233 cm⁻¹ referred to the C—N stretch and C—N—H bend in amide III β sheets structure (Hu et al. (2006) Macromolecules 39(18):6161-6170; Hayashi et al. (2007) J. Phys. Chem. B 111(37):11032-11046). Two strong peaks were shown at the regions of 1619-1622 cm⁻¹ for C═O stretch of amide I and 1509-1516 cm⁻¹ for the C—N stretch and C—N—H bend for amide II (Horan et al. (2005) Biomaterials 26(17):3385-3393) (FIG. 1). Type I collagen had a characteristic broad peak at the absorption of 3275 cm-1 which was indicative for the —O—H stretch and N—H stretch in this region (FIG. 1—region 1). PGS polymer had a distinct FTIR spectrum which showed a broad —OH stretch at 3458 cm⁻¹. This characteristic was reported to indicate the hydrogen bonded hydroxyl groups in PGS (Wang et al. (2002) Nat. Biotech. 20(6): 602-606). Sharp transmittance peaks at 2928 cm⁻¹ and 2855 cm-1 (FIG. 1—region 2) were shown to represent the sp3 C—H stretch (asymmetry and symmetry). Moreover, the intense ester C═O stretch at the absorption of 1734 cm⁻¹ (FIG. 1—region 3) was another unique feature for PGS which was not presented in either collagen or fibroin. Amide III was at the absorption range of 1228-1237 cm⁻¹ (FIG. 1—region 6). Amide I, II were present at the absorption range of 1622-1630 cm⁻¹ (FIG. 1—region 4) and 1546-1515 cm⁻¹ (FIG. 1—region 5) respectively. In summary, all composites were demonstrated to be blends of strategy materials based on identification of expected absorption wavenumbers (FIG. 1).

### Example 2

Fiber Morphology.

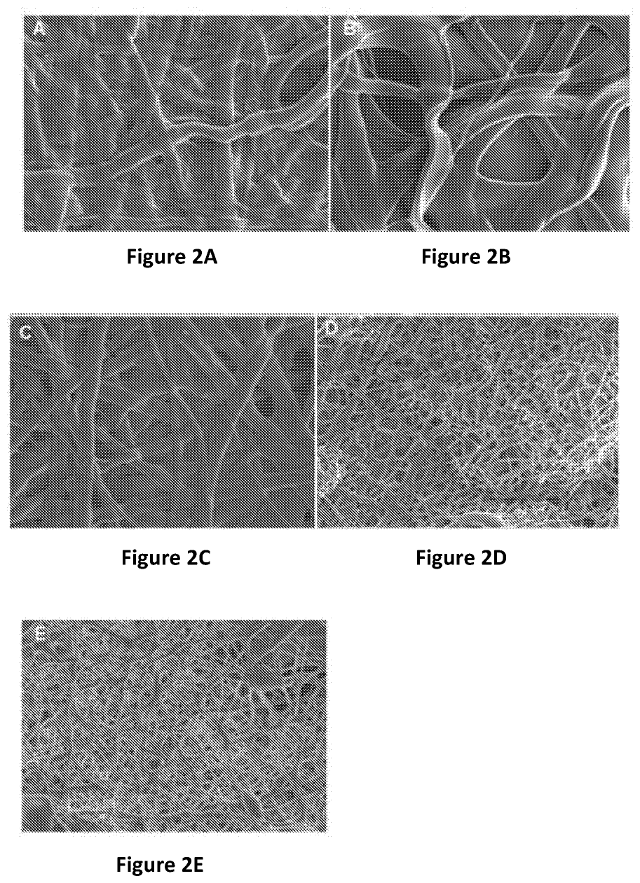

Fiber diameters ranged from 694 to 4577 nm (Table 1). In general, thinner and more rounded fibers were observed for the electrospun mats with higher fibroin content (90% and 80%) as compared to the thicker and more flat fiber of electrospun mats with high proportions of collagen (90%, 80%, and 45%). The interconnected fiber network structures of electrospun mats at various collagen, fibroin and PGS weight ratios were compared after crosslinking using scanning electron microscopy (SEM) (FIG. 2).

TABLE 1
Fiber Diameters in Mats of Different Compositions

| Sample Type | Fiber Diameters (nm) |
|---|---|
| Collagen:PGS (9:1) | 2067 ± 168 |
| Collagen:Fibroin:PGS (8:1:1) | 4577 ± 697 |
| Collagen:Fibroin:PGS (4.5:4.5:1) | 2952 ± 240 |
| Collagen:Fibroin:PGS (1:8:1) | 784 ± 77 |
| Fibroin:PGS (9:1) | 694 ± 43 |

All values represent means ± SEM;
The fiber diameters were measured from 16 randomly selected fibers of two representative SEM pictures. The measurements are presented as mean ± standard error of the mean.

### Example 3

Thermal Transition Analysis of Electrospun Mats.

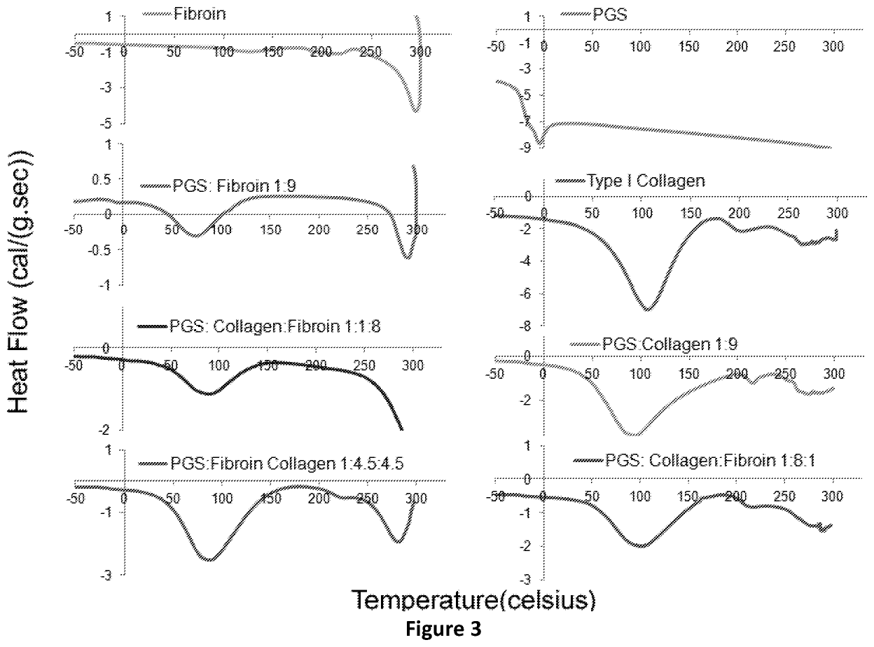

Results of DSC scan were analyzed to determine thermal transition temperatures of electrospun mats (FIG. 3). By incorporating an increasing amount of fibroin, a shift of thermal transition temperature to higher range was observed. The electrospun composite materials exhibit much higher thermal transition temperatures as compared to PGS alone. Results suggest the electrospun mats made from collagen, silk fibroin, and PGS composites were thermally stable for in vivo application.

### Example 4

Porosity Measurements.

Porosity of the electrospun mats is important for nutrient and oxygen transport as well as cell adhesion and proliferation. Triplicate samples of all mats were weighted to determine dry weight. The porosity of the electrospun mats ranged from 67% to 80% (n=3) which is a sufficiently large surface and internal area for cell adhesion, migration and nutrients transport.

### Example 5

Mechanical Tensile Testing.

An essential feature of heart valve materials is the tensile and elastic properties. The elastic moduli of the electrospun mats (Table 2) ranged from 2.25 Mpa (Collagen:PGS=9:1) to 4.97 Mpa (Fibroin:PGS=9:1). The highest elastic modulus was observed for composites containing 45 or 80% fibroin. Electrospun mats with collagen alone had a similar elastic modulus of 3.67 Mpa compared to collagen-based porcine valvular grafts: (3.68 MPa fresh valves and 3.95 MPa) glutaraldehyde fixed valves (Vesely et al. (1992) J. Biomech. 25(1):101-113). Incorporation of PGS created graft materials with customized elastic propertied with strain ranged from 30-70%. All mats produced had stress values between 0.69 Mpa and 1.45. These values were 100-fold greater than stress exerted by blood pressure under normal or hypertensive states. Therefore, the electrospun mats have sufficient mechanical properties to withstanding blood pressure effects. Among all electrospun materials, the composites at 4.5:4.5:1 of collagen, fibroin and PGS weight ratio had the best overall mechanical strength and elasticity.

TABLE 2
Uniaxial Tensile Testing of Electrospun Mats

| Sample Type | Elastic Modulus (Mpa) | Stress (Mpa) | Strain (mm/mm) |
|---|---|---|---|
| Collagen | 3.67 ± 0.12^{a} | 0.69 ± 0.0^{6a4} | 0.23 ± 0.03^{a d} |
| Collagen:PGS (9:1) | 2.25 ± 0.16^{b} | 1.30 ± 0.0^{9b,c} | 0.62 ± 0.02^{b} |
| Collagen:Fibroin:PGS (8:1:1) | 2.76 ± 0.20^{b} | 1.10 ± 0.0^{9b} | 0.44 ± 0.03^{b,c} |
| Collagen:Fibroin:PGS (4.5:4.5:1) | 4.11 ± 0.13^{a,c} | 1.45 ± 0.0^{5c} | 0.41 ± 0.01^{a,b,c} |
| Collagen:Fibroin:PGS (1:8:1) | N/A: too brittle to be determined | | |
| Fibroin:PGS (9:1) | 4.97 ± 0.27^{d} | 0.82 ± 0.0^{9d} | 0.33 ± 0.12^{a,c} |

All values represent means ± SEM
The tensile stress, strain and elastic modulus of electrospun mats at different composite ratios were measured. Data are presented as mean ± Standard error of the mean (n = 3). Numbers designated with the same letter are not significantly different, whereas numbers with different letters are significantly different (p < 0.05).

### Example 6

Modified Suture Retention Strength Testing.

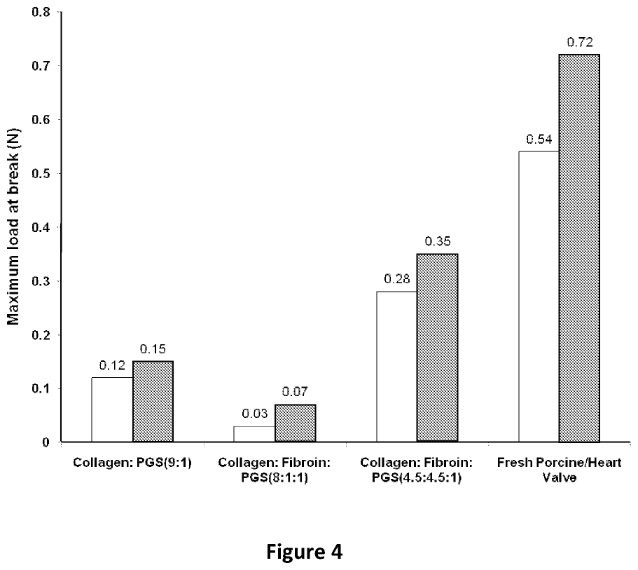

The suture retention test was used to assess the maximum force required to disrupt sutures from materials (FIG. 4). Collagen, fibroin, and PGS electrospun mats at 4.5:4.5:1 weight ratio required the greatest suture retention force with a maximum load of 0.32N, which is closest to the suture retention force of fresh porcine heart valve (0.64N) as compared to other electrospun composites.

Based on the mechanical and physical properties, the electrospun mats containing collagen-fibroin-PGS (4.5:4.5:1 weight ratio) (PFC mats) were most similar to the mechanical properties of fresh aortic valve. The PFC mats were studied further to evaluate the degradation and cellular compatibility using endothelial cells and blood platelets. Thrombogenicity studies included cell growth on the material, formation of a tight monolayer, while thrombogenicity was assessed using by cell compatibility test the interaction of platelets.

### Example 7

Degradation of PFC Mats.

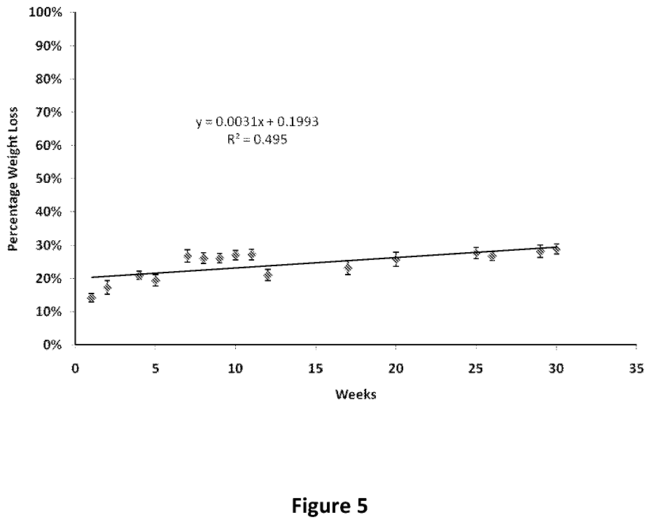

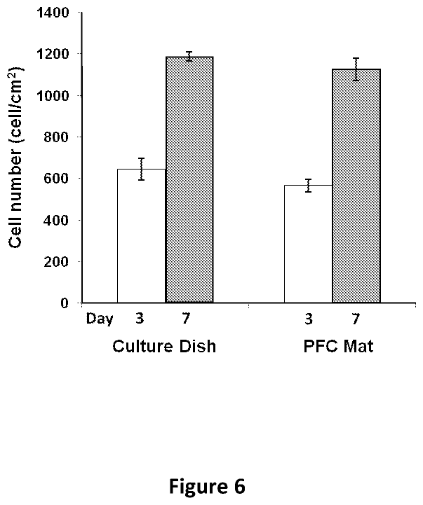

Degradation of PFC mats was found to be only a 0.3% weight loss over a 30 week incubation period (FIG. 5). With the unaided eye, PFC mats were observed to remain intact during the entire degradation time period. SEM was used to examine the fine structure of nanofibers. A consistent morphology of the fibers was observed within the fiber meshwork and was unchanged during the course of study. Fiber diameter measurements on mats indicated no significant differences over the course of the experiment.

### Example 8

Cell Adhesion and Proliferation Study—Cell Compatibility Test.

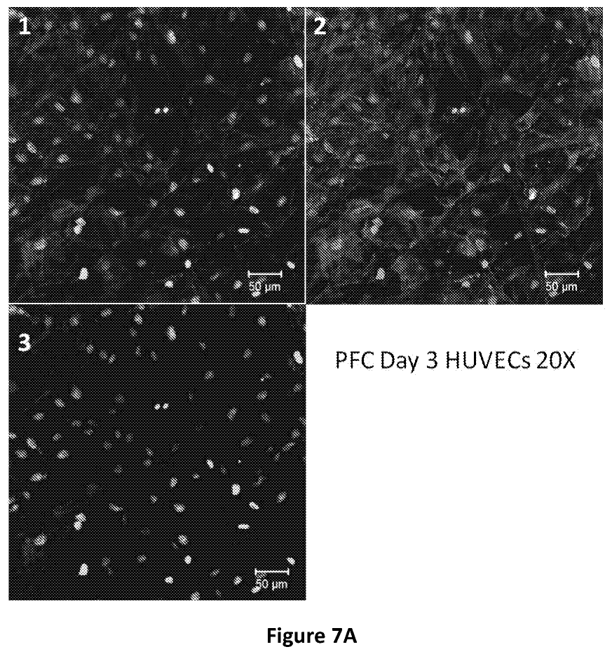

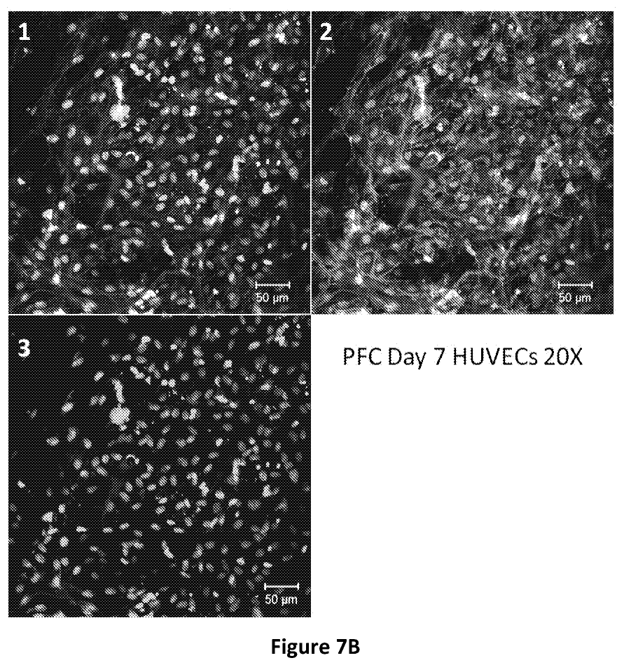

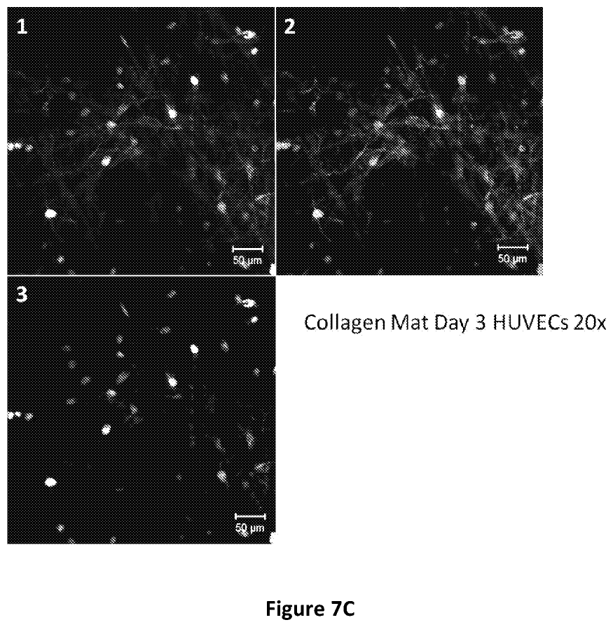

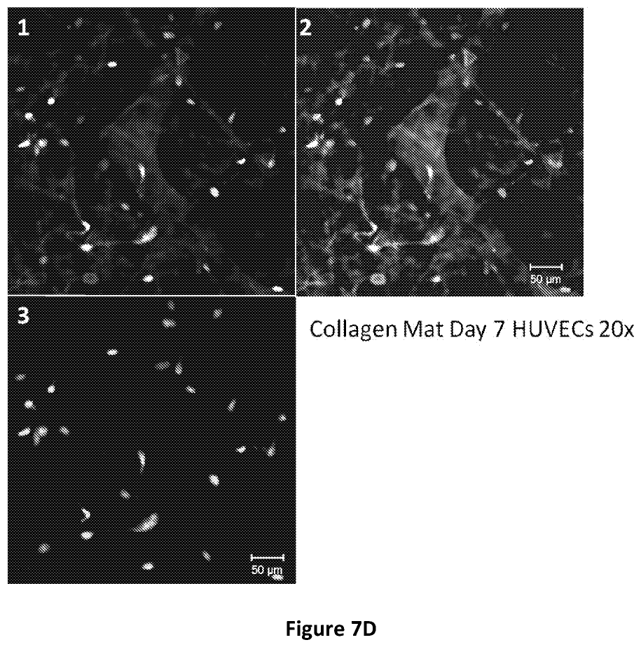

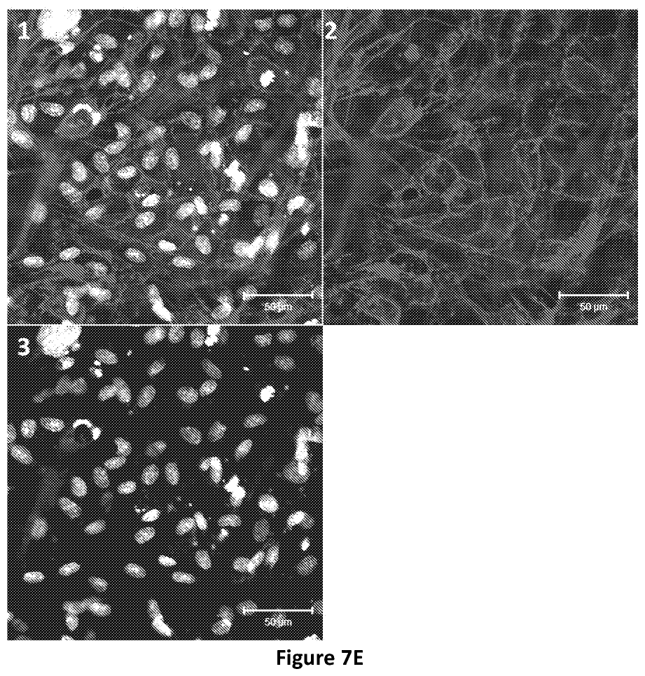

Biocompatibility and the formation of a monolayer of endothelial cells on blood contacting biomaterials is a necessary component to provide a functional and non-thrombogenics surface. When HUVEC's were cultured on PFC mats, cell numbers increased significantly (p<0.05) from day 3 to day 7 (FIG. 6) Morphologically, HUVECs on culture dishes appeared evenly spread without significant cell-cell interactions, whereas on PFC mats, an organized uniform cell sheet was formed (FIG. 7). The intense staining pattern of F-actin at the cell borders for cells cultured on PFC mats suggested the formation of tight junctions (FIGS. 7A and 7B).

In contrast, when HUVEC's were cultured on collagen mats, seeded with the same number of cells as the PFC mats, the endothelial cells were not observed to form tight junctions (FIGS. 7C and 7D).

Upon examining the cells at a higher magnification, areas demonstrating tight cell-cell interactions were observed for cells cultured on PFC mats (FIG. 7E). As reported, F-actin indirectly binds to the endothelial tight junction protein such as VE-cadherin along the cell-cell junction. The intense staining pattern of F-actin at the cell borders suggests the formation of tight junctions.

### Example 9

Platelet Adhesion to PFC Mats—Quantifying Adhered Platelets on Various Substrates.

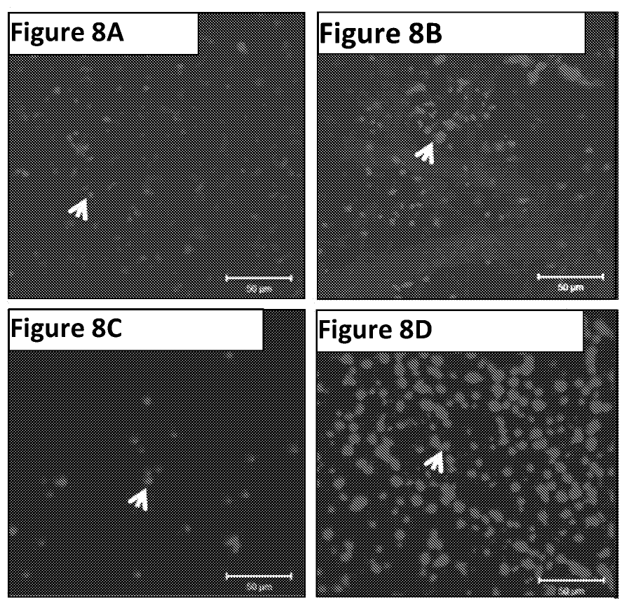

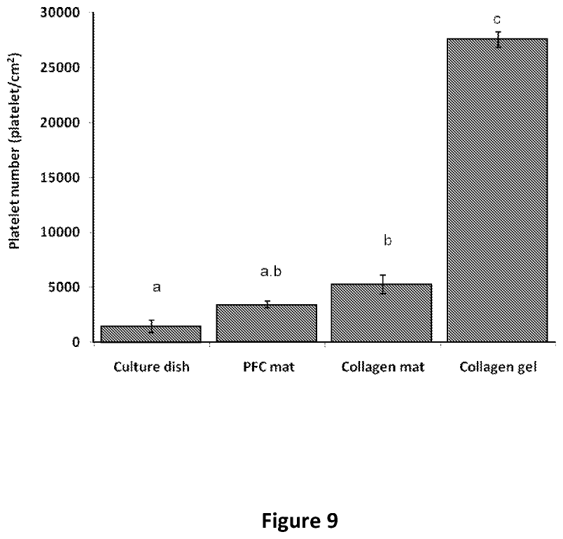

Several studies were completed using platelets to assess the thrombogenic nature of the PFC mats alone or of mats cultured with cells. Confocal images of platelets stained with rhodamine-phalloidin demonstrated a low level of adhesion of platelet on polysterene culture dishes (FIG. 8). Single platelets or small clumps comprised of 2-3 platelets were present on PFC mats. In sharp contrast, platelets and platelet aggregates adhered extensively to the collagen gel (positive control) where images revealed increased platelets numbers as well as increased sizes of aggregates (FIG. 8). Fewer platelets and fewer platelet clumps were observed on PFC mats as compared to electrospun collagen nanofiber mats as compared to collagen controls. The numbers of adherent platelets on various substrates were determined by using cell counter in Image J software (FIG. 9). Platelet numbers on collagen mat, and collagen gel were respectively 1.5 and 7.9 fold higher (p<0.05) than on the PFC mats.

### Example 10

Platelet Adhesion to PFC Mats—Morphological Identification of Platelets Activation.

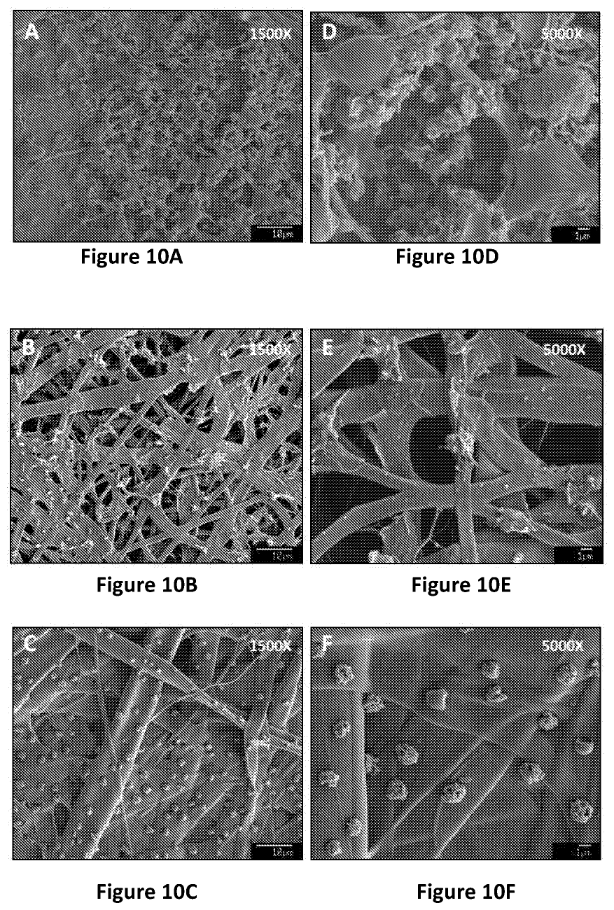

Platelet morphology was examined using SEM to evaluate the degree of platelet activation (FIG. 10). The results demonstrate extensive activation and fused degranulated platelets for platelets on collagen gels (FIGS. 10A and 10D). Platelets were more activated on electrospun collagen mats than PFC mats. (FIGS. 10B and 10E) Platelets on PFC mats appeared less activated with distinctly spherical features (FIGS. 10C and 10F) as compared to the appearance of platelets on collagen electrospun mat on the collagen gel.

### Example 11

Platelet Adhesion to Materials Containing HUVECs.

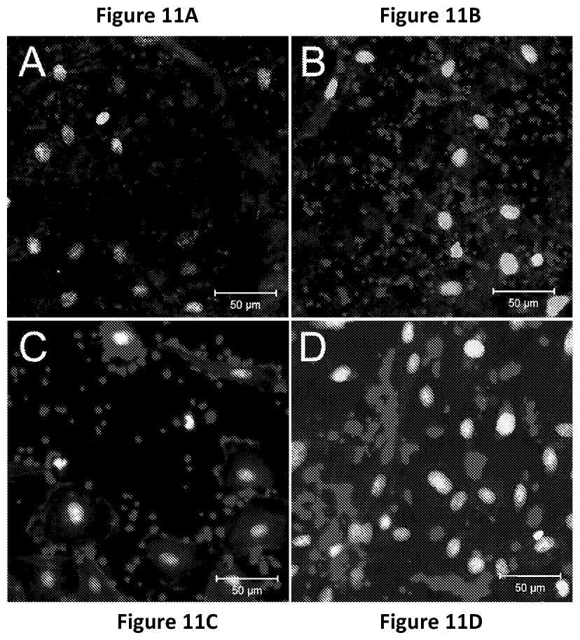

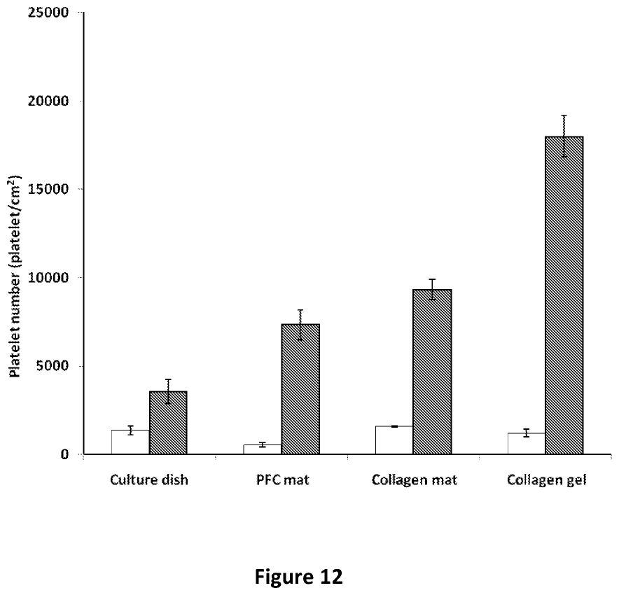

In a second experiment platelets were applied to mats cultured for 24 hours with HUVECs (50,000 cells/well in a 48-well plate) (FIG. 11). More adherent platelets on collagen electrospun mat and formation of microthrombi in larger size on collagen gel were seen, in contrast to single platelets or clumps of 2-3 platelets on culture dishes and PFC mats (FIG. 11). For all materials the presence of endothelial cells significantly reduced the number of adherent platelets demonstrating the nonthrombogenic nature of the endothelial cell glycocalyx. A 60% reduction in total adherent platelets mat was observed (FIG. 12) on the PFC mat as compared to the culture dish (p<0.05). The number of platelets decreased 2.9-fold on PFC mat compared to the electrospun collagen mat (p<0.05), and 2.2 fold compared to the collagen gel (p<0.05).

When areas of mats without endothelial cells were examined, while not statistically significant, a 27% reduction of platelet number on the PFC mat was observed as compared to the electrospun collagen (FIG. 12).

### Example 12

Autologous saphenous vein is the standard material for bypassing small diameter (<6 mm) coronary arteries, but is subject to intimal hyperplasia, thrombosis, and accelerated atherosclerosis. To date, no biomaterial functions as a substitute for vein graft. Herein, an endovascular biomaterial, PFC, composed of electrospun poly-(glycerol sebacate), silk fibroin, and type 1 collagen has been developed. The biomaterial has tensile, non-thrombogenic, and endothelial cell adhesive properties ideal for use as an artery graft material. Here, a conduit from PFC was fine tuned with cell derived extracellular matrix (ECM) and cell growth and mechanical properties of the conduit were tested. The addition of ECM components onto the PFC material strengthens the material and deposits molecules that facilitate cellular adhesion and proliferation.

Materials and Methods:

PFC-PGS: Silk Fibroin: Type I Collagen at 1:4.5:4.5. Electrospinning conditions: 10% PFC in 1,1,1,3,3,3-hexafluoro-2-propanol (HFP). PFC graft was fabricated by electrospinning 1 ml of polyethylene glycol (PEG) and 2.3 mls of PFC onto a 3.4 mm diameter stainless steel dowel. Sacrificial PEG layer was removed through a series of washes (See Example 13).

Sparse PFC mats composed of nanofibers were fabricated by electrospinning 0.2 mls of PFC onto tissue culture plastic treated slides, resulting in a sparse network of PFC fibers. NIH 3T3 mouse fibroblasts were cultured on the PFC material for 6 days and decellularized using Trypsin/Triton X-100.

Dense PFC mats were fabricated by electrospinning 4.4 mls of PFC onto a 9 by 10 cm rectangle of aluminum foil. NIH 3T3 mouse fibroblasts were seeded on material for 10 days and either immediately fixed (n=3) or decellularized (n=3) and fixed. All PFC materials were heat cured at 120° C. for 48 hours and crosslinked with glutaraldehyde vapor for 24 hours.

Porous PFC mats were fabricated by electrospinning equal volumes of PFC and poly(ethylene oxide) (PEO). Material was subsequently glutaraldehyde crosslinked, PEO was leached, and material was heat treated. Human aortic smooth muscle cells (HASMCs) were seeded on material for 7, 14, 21 days and fixed and imaged to determine infiltration.

Results and Discussion:

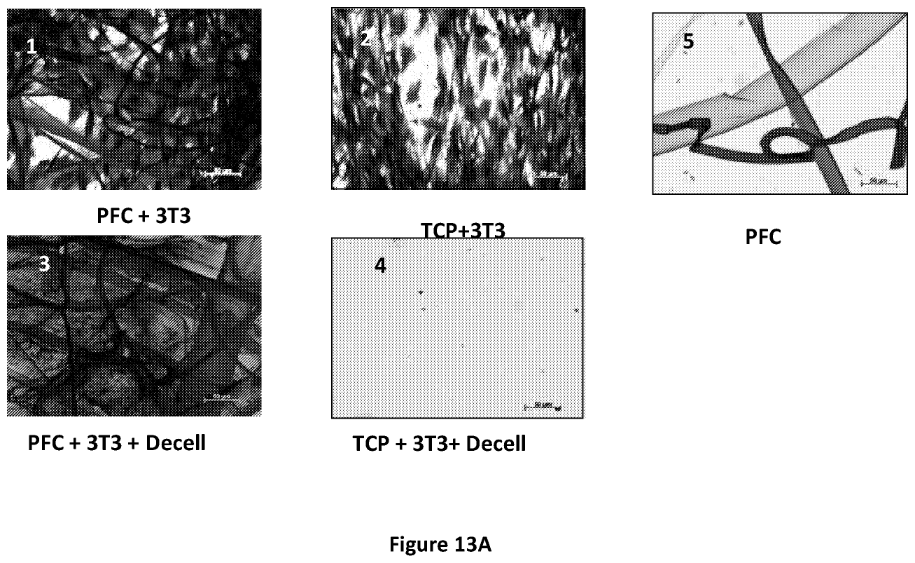

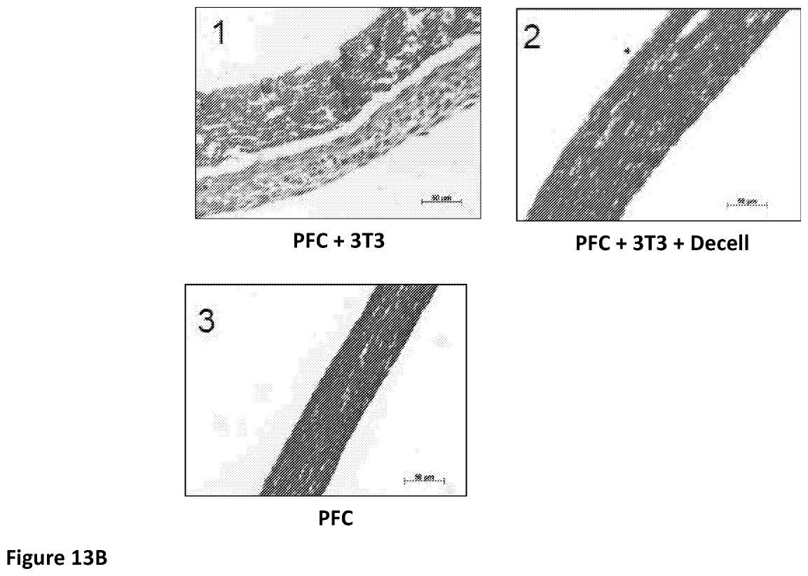

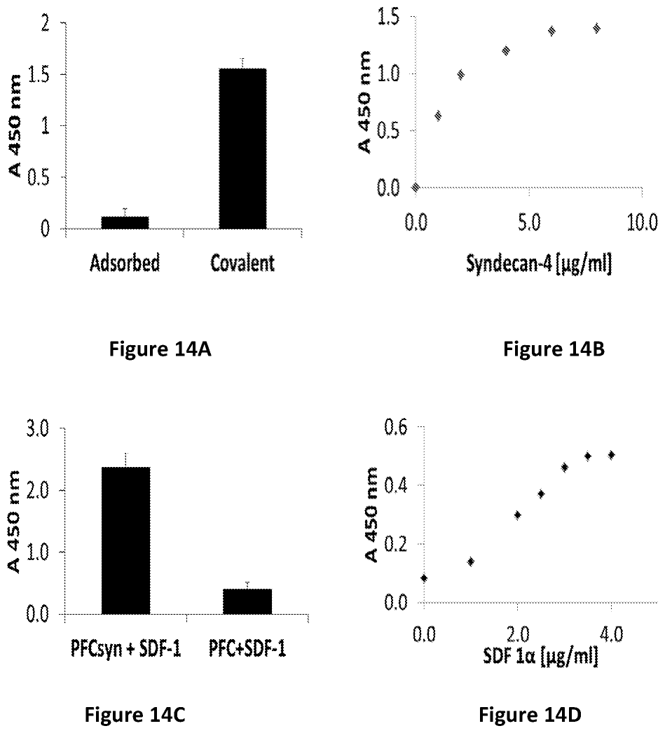

Fibroblasts deposited a nanofibrous ECM on the surface of sparsely electrospun PFC as shown by crystal violet staining (FIG. 13A). The ECM was apparent on sparse PFC after decellularization with Triton X-100. The PFC facilitated the ability for the ECM to remain on the material compared to tissue culture plastic slides without PFC. Fibroblasts formed a dense monolayer on the surface of PFC but did not infiltrate the material. A porous PFC material was fabricated to support cellular infiltration. Porous PFC supported proliferation and infiltration of HASMCs, cells which also produce extracellular matrix. This shows that the properties of not just the surface but the interior of the PFC material can be tuned. FIG. 13B provides light micrographs of H&E stained 3T3 fibroblasts cultured on dense PFC mats, 3T3 fibroblasts cultured on dense PFC mats and decellularized with Triton X-100, and dense PFC mats without cells cultured.

Conclusions:

These studies show 1) a graft material composed of PFC can be synthesized, 2) it is possible to effectively decellularize PFC seeded with fibroblasts and maintain ECM material, and 3) the ability to potentially tune the properties of the material.

### Example 13

PFC/PEO Fabrication Protocol

Day 1
1. Melt PGS prepolymer at 80° C. in oven for ˜30 minutes, measure out 0.05 g and allow to harden.
2. Add PGS, 0.225 g fibroin, 0.225 Type 1 collagen to jar with stir bar, add 5 mls HFIP.
3. Tightly close car and seal with parafilm. Stir for 24 hours on corning stir plate setting 3.

Day 2
1. Add 0.5 g PEO to 5 ml 90% EtOH with 0.01% Crystal Violet in jar with stirbar, seal jar, stir for 6 hours on corning stir plate setting 3.
2. Set up electrospinning box with rotating motor with steel pipe flanked by 2 elevated syringe pumps.
3. Add dissolved PEO and PFC solutions to syringes with blunt tip needles. Bend needs to 90.
4. Set syringe pumps to 2 ml/hr.
5. Connect wiring so ground is connected to motorized pump grounding coil and both voltage clips are connected to syringe pump needle of PEO solution.
6. Make sure tips of needles are aligned with steel pipe and are 18 cm away from edge of pipe.
7. Turn on PEO solution and PFC solution syringe pumps.
8. Turn on vertical oscillating and rotating mandrel motor.
9. Set voltage to 25 kV and turn on voltage source.
10. Electrospin 3.5 mls of PFC at the same time as PEO.
11. Turn off voltage box, ground everything.
12. Remove PFC/PEO electrospun material with pipe and put into glutaraldehyde chamber with glutaraldehyde and water (3.2 mls 25% glutaraldehyde in 4.8 mls ddH₂O). Crosslink for 24 hours.

Day 3
1. Remove PFC material from chamber. Wash material in 0.02M glycine for 30 minutes×2.
2. Remove PFC material from stainless pipe by cutting with scaple.
3. Wash material 15 minutes×4 in 70% EtOH.
4. Wash material in H₂O 15 minutes ×2.
5. Dry overnight at RT in a vacuum oven set to 5 mmHg on Teflon sheet.

Day 4
1 Place PFC on Teflon sheet in 120° C. oven for 48 hours.

Day 6
1. Remove from oven. Store material in vacuum chamber at RT until use.

### Example 14

Autologous saphenous vein is the standard material for bypassing small diameter (<6 mm) coronary arteries, but is subject to intimal hyperplasia, thrombosis, and accelerated atherosclerosis. To date, no biomaterial functions as a substitute for vein graft. Herein, an endovascular scaffold biomaterial, PFC, composed of electrospun poly-(glycerol sebacate), silk fibroin, and type 1 collagen has been developed. The biomaterial has tensile, nonthrombogenic, and endothelial cell adhesive properties ideal for use as an artery graft material. The cytokine SDF-1α/CXCL12 has been shown to localize endothelial progenitor cells to areas of ischemia by binding to the CXCR4 receptor but has a relatively short half-life in the bloodstream. Syndecan 4 (SD-4) is a high affinity receptor for SDF-1α. Herein, SD-4 is covalently bound to the PFC scaffold in order to immobilize SDF-1α and promote endothelial cell recruitment. This construct results in accelerating the formation of an endothelial monolayer on vascular grafts of PFC.

Methods

Preparation of PFC Materials:

PFC materials were fabricated by electrospinning a 10% w/v ratio of 1:4.5:4.5 PGS: Silk Fibroin: Type 1 collagen in 1,1,1,3,3,3, hexafluoroisopropanol. PFC mats were subsequently heat treated at 120° C. for 48 hours and treated with glutaraldehyde vapor for 24 hours to cure PGS and crosslink collagen fibers, respectively.

Covalent Linkage of SD-4 to PFC Materials:

PFC scaffolds were incubated in 2-(N-morpholino)ethanesulfonic acid buffer (MES) (50 mmol) for 30 minutes at room temperature. Scaffolds were then incubated in 1-ethyl-3-(3-dimethylaminopropyl) carbodiimide (EDC) (30 mmol) and n-hydroxysuccinimide (NETS) (6 mmol) in MES buffer for 30 minutes at room temperature followed by rinsing with MES buffer. Scaffolds were incubated with SD4 (2 μg/ml) in MES buffer for 2 hours at 37° C. with agitation. PFC scaffolds adsorbed with SD4 (2 μg/ml) were used as a comparison. To determine the loading capacity of SD4 on PFC, PFC was incubated in increasing concentrations of SD4 during conjugation.

ELISA Assay to Determine SD4:

Scaffolds were rinsed with phosphate buffered saline (PBS) and blocked with 0.1% casein in PBST (PBS+0.05% Tween-20) overnight in at 4° C. Wells were incubated with primary antibodies against anti-SD4 diluted in 0.1% casein in PBST for 2 hours at room temperature. Samples were rinsed with PBS with 0.05% Tween 20 and incubated with horseradish peroxidase (HRP) linked secondary antibodies diluted in 0.1% casein in PBST. HRP substrate 3,3′,5,5′-Tetramethylbenzidine (TMB) was added for 5-30 minutes and stopped with a solution of 2 M sulfuric acid. Materials were removed from wells and remaining solution was read at 450 nm.

Immobilization of SDF-1α on Modified PFC Scaffolds:

PFC scaffolds with covalently bound SD4 were fabricated as described above. Scaffolds without SD4 were used as a control. All scaffolds were rinsed with PBS and added to SDF-1α (2 μg/ml) for 2 hours at 37° C. with agitation. The amount of bound SDF-1α was measured by ELISA analysis as described previously, using anti-SDF-1α as the primary antibody. To determine the loading capacity SDF-1α on SD4 conjugated PFC (PFCSYN), PFCSYN was incubated in increasing concentrations of SDF-1α during conjugation.

Results

There was significantly more SD4 present on materials after covalent linkage than on materials adsorbed with SD4 (p<0.05). Saturation of SD4 on PFC materials occurred around 2 μg/ml of SD4. There was an increased ability of SDF-1α to adsorb to materials conjugated with SD4 compared to materials without SD4 (p<0.05). Saturation of SDF-1α on the PFC materials covalently linked to SD4 occurred around 3 μg/ml of SDF-1α.

The functionalized PFC scaffold will home circulating stem cells once the PFC is surgically implanted. Results of this study demonstrate that SD-4 can be covalently linked to the PFC scaffold material and sequester the growth factor SDF-1α despite being covalently crosslinked to the scaffold material (i.e., retains its biological activity).

### Example 15

PFC was functionalized by covalently linking signaling molecules to the surface via carbodiimide chemistry. Crosslinking PFC may decrease the amount of carboxyl groups available for further functionalization. Herein, crosslinking conditions for further functionalization of the PFC material are determined.

Materials and Methods

Preparation of PFC Materials:

PFC materials were fabricated by electrospinning a 10% w/v ratio of 1:4.5:4.5 PGS: Silk Fibroin: Type 1 collagen in 1,1,1,3,3,3, hexafluoroisopropanol. PFC mats were subsequently heat treated at 120° C. for 48 hours to polymerize the PGS component.

Crosslinking Conditions:

Materials were either crosslinked with 0.5, 1.5, 5, or 10% glutaraldehyde vapor for 24 hours or with 1-ethyl-3-(3-dimethylaminopropyl) carbodiimide (EDC) (30 mmol) and N-hydroxysuccinimide (NHS) (6 mmol) in ethanol for 5, 15, 30, or 60 minutes.

Fiber Morphology:

The morphology of the fibers was characterized by scanning electron microscopy (SEM).

Endothelial Cell Proliferation:

Human umbilical vein endothelial cells (HUVECs) were cultured on PFC materials at a density of 5×10³ cells/cm² in EGM-2 media. Proliferation of cells on crosslinked PFC was measured by MTS assay at days 1, 3, and 5.

Free Amine Group Determination:

Reactive amine groups remaining on the materials were determined using 2,4,6-trinitrobenzenesulfonic acid (TNBSA).

Results

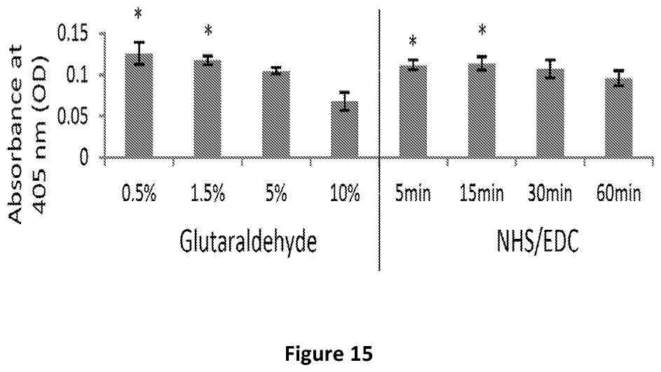

SEM images of the crosslinked material show consistent fiber morphologies with the exception of fibers crosslinked for 5 minutes in EDC/NHS. These fibers appear indistinct, indicating inadequate stabilization. Based on MTS data, cells continuously proliferated on fibers crosslinked for 15, 30, and 60 minutes with NHS/EDC as well as with 0.5% glutaraldehyde. Cells show minimal proliferation on fibers crosslinked for 5 minutes in NHS/EDC. Cells on fibers crosslinked with 1.5, 5, and 10% glutaraldehyde had decreased proliferation from days 3 to 5, suggesting cytotoxicity of glutaraldehyde fixed scaffolds. There are significantly less reactive amine groups in materials crosslinked with 10% glutaraldehyde compared to fibers crosslinked with 0.5% and 1.5% glutaraldehyde and NHS/EDC for 5 and 15 minutes. A trend appears to show less reactive amino groups with increasing crosslinked time or glutaraldehyde concentration (FIG. 15).

Increasing the crosslinking of PFC fibers decreases the availability of reactive groups for further functionalization. Based on cell response, fiber morphology, and reactive amine groups, PFC crosslinked for 15 minutes with NHS/EDC or with 0.5% glutaraldehyde appeared superior.

A number of patent and non-patent publications are cited in this application in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope of the appended claims.

Furthermore, the transitional terms “comprising”, “consisting essentially of” and “consisting of”, when used in the appended claims, in original and amended form, define claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified materials (s). The term “consisting essentially of” limits the scope of a claim to the specified elements, steps or material(s) and those that do not material affect the basic and novel characteristic (s) of the claimed invention. All biomaterials and methods for preparing and utilizing the same that embody the present invention can, in alternate embodiments, be more specifically defined by any of the transitional terms “comprising”, “consisting essentially of” and “consisting of”.

The singular forms “a,” “an,” and “the” include plural referents unless the context clearly dictates otherwise.

The term “thrombogenicity” refers to the tendency of a material in contact with blood to produce a clot or thrombus. A nonthrombogenic surface is a surface that tends not to cause the formation of a clot or thrombus when brought into contact with blood, particularly under physiological conditions (e.g., in a blood vessel). For example, the surface may be less thrombogenic than collagen.

1: An electrospun fiber material comprising a collagen, a fibroin, and a hemocompatible synthetic elastomer,
wherein said electrospun nanofiber material further comprises extracellular matrix and/or a heparan sulfate glycoprotein.

2: The electrospun fiber material of claim 1 comprising a heparan sulfate glycoprotein.

3: The electrospun fiber material of claim 1 comprising extracellular matrix.

4: The electrospun fiber material of claim 2, wherein said heparin sulfate glycoprotein is covalently linked to said electrospun fiber material.

5: The electrospun fiber material of claim 4, wherein said heparin sulfate glycoprotein is a syndecan.

6: The electrospun fiber material of claim 5, wherein said syndecan is syndecan-4.

7: The electrospun fiber material of claim 1, further comprising stromal cell-derived factor 1 (SDF-1α).

8: The electrospun fiber material of claim 1, wherein the collagen is type I collagen.

9: The electrospun fiber material of claim 1, wherein the fibroin is silk fibroin.

10: The electrospun fiber material of claim 1, wherein the hemocompatible synthetic elastomer is a poly glycerol derivative ester comprising a polycarboxylic acid.

11: The electrospun fiber material of claim 1, wherein the hemocompatible synthetic elastomer is poly (glycerol sebacate).

12: The electrospun fiber material of claim 1, comprising type I collagen, silk fibroin, and poly (glycerol sebacate).

13: The electrospun fiber material of claim 1, wherein the weight ratio for the collagen is about 25% to about 65% per total composition weight of said collagen, fibroin, and hemocompatible synthetic elastomer.

14: The electrospun fiber material according to claim 13, wherein the weight ratio for the collagen is about 45% per total composition weight of said collagen, fibroin, and hemocompatible synthetic elastomer.

15: The electrospun fiber material of claim 1, wherein the weight ratio for the fibroin is about 25% to about 65% per total composition weight of said collagen, fibroin, and hemocompatible synthetic elastomer.

16: The electrospun fiber material according to claim 15, wherein the weight ratio for the fibroin is about 45% per total composition weight of said collagen, fibroin, and hemocompatible synthetic elastomer.

17: The electrospun fiber material of claim 1, wherein the weight ratio for the hemocompatible synthetic elastomer is about 10% to about 20% per total composition weight of said collagen, fibroin, and hemocompatible synthetic elastomer.

18: The electrospun fiber material according to claim 17, wherein the weight ratio for the hemocompatible synthetic elastomer is about 10% per total composition weight of said collagen, fibroin, and hemocompatible synthetic elastomer.

19: The electrospun fiber material of claim 1, wherein the weight ratio for the hemocompatible synthetic elastomer is about 10%, the weight ratio for the collagen is about 45%, and the weight ratio for the fibroin is about 45% per total composition weight of said collagen, fibroin, and hemocompatible synthetic elastomer.

20: The electrospun fiber material of claim 1, further comprising an electronegative carbohydrate, a glycosaminoglycan, a growth factor, a cell signaling molecule, or a combination thereof.

21: The electrospun fiber material of claim 1, wherein the fibers have a diameter of about 600 to about 5000 nm.

22: A porous electrospun graft material configured to replace biological tissue comprising the electrospun fiber material of claim 1.

23: The porous electrospun graft material according to claim 22, wherein the graft material is shaped as a heart valve, an aortic heart valve, a stent, a sheet, or a conduit.

24: The porous electrospun graft material according to claim 22, wherein the graft material is shaped as an arterial or venous conduit, particularly with a diameter less than 6 mm.

25: The porous electrospun graft material according to claim 22, wherein the graft has an elastic modulus from about 2 to about 5 Mpa.

26: The porous electrospun graft material according to claim 22, wherein the graft has a stress value of about 0.6 to 2.0 Mpa.

27: The porous electrospun graft material according to claim 22, wherein the graft has a strain value of about 0.2 to 0.7 mm/mm.

28: A method of preparing the electrospun fiber material of claim 1, the method comprising the steps of:
a. electrospinning a solution comprising collagen, fibroin and a hemocompatible synthetic elastomer into an interconnected fiber matrix;
b. culturing the interconnected fiber matrix of step a) with extracellular matrix producing cells; and
c. collecting the interconnected fiber matrix, thereby preparing said electrospun fiber material.

29: The method of claim 28, wherein step (a) comprises electrospinning the solution onto a mold.

30: The method of claim 28, further comprising the step of curing the interconnected fiber matrix of step a) by heating the interconnected fiber matrix and/or crosslinking.

31: The method of claim 28, wherein the extracellular matrix producing cells are fibroblasts.

32: The method of claim 28, wherein the interconnected fiber matrix is cultured with said extracellular matrix producing cells for about 1 to about 30 days.

33: The method of claim 28, further comprising lysing the cultured cells by delivering a lysing agent to the cells cultured with the interconnected fiber matrix.

34: The method of claim 33, wherein said lysing agent is a non-ionic detergent.

35: A method of preparing the electrospun fiber material of claim 1, the method comprising the steps of:
a. electrospinning a solution comprising collagen, fibroin and a hemocompatible synthetic elastomer into an interconnected fiber matrix;
b. crosslinking said heparan sulfate glycoprotein to the interconnected fiber matrix of step a), thereby preparing said electrospun fiber material.

36: The method of claim 35, wherein step b) is performed with a carbodiimide crosslinker.

37: The method of claim 35, further comprising the step of curing the interconnected fiber matrix of step a) by heating the interconnected fiber matrix and/or crosslinking prior to step b).
